SEQUENCE LISTING (2 sequences)

SEQ ID NO: 1 - protein, 21 aa, Homo sapiens
GIVEQCCTSICSLYQLENYCN

SEQ ID NO: 2 - protein, 30 aa, Homo sapiens
FVNQHLCGSHLVEALYLVCGERGFFYTPKT

(19) United States
(12) Patent Application Publication
(10) Pub. No.: US 2017/0254798 A1
(43) Pub. Date: Sep. 7, 2017

(54) METHOD FOR SCREENING DRUG-SACCHARIDE CONJUGATES

(71) Applicant: Merck Sharp & Dohme Corp., Rahway, NJ (US)

(72) Inventors: Yingjun Mu, Fanwood, NJ (US); Ruojing Yang, Boston, MA (US); Xinghai Li, Torrance, CA (US); Margaret Wu, Scotch Plains, NJ (US); Qing Dallas-yang, Westfield, NJ (US); Yuli Chen, Scotch Plains, NJ (US); Ge Dai, Edison, NJ (US); Songnian Lin, Monroe, NJ (US); David E. Kelley, Westfield, NJ (US); Mark Erion, Westfield, NJ (US)

(73) Assignee: Merck Sharp & Dohme Corp., Rahway, NJ (US)

(21) Appl. No.: 15/060,786

(22) Filed: Mar. 4, 2016

(51) Int. Cl. G01N 33/50; G01N 33/74

(52) U.S. Cl. CPC G01N 33/502; G01N 33/74; G01N 2333/70596; G01N 2333/62; G01N 2500/04; G01N 2500/20; G01N 2400/00

(57) ABSTRACT

Methods for identifying drug-saccharide conjugates that bind the mannose receptor, C type 1, (MRC1) and may be taken up by cells expressing MRC1, and the use of such drug-saccharide conjugates for treatment of particular diseases are described. In particular, the methods for identifying insulin-saccharide conjugates that bind the MRC1 receptor and may be taken up by cells expressing the MRC1 and use of such conjugates for treatment of diabetes are described.

### REFERENCE TO SEQUENCE LISTING SUBMITTED ELECTRONICALLY

The sequence listing of the present application is submitted electronically via EFS-Web as an ASCII formatted sequence listing with a file name “23925-US-NP-SEQTXT-04MAR2016.txt”, creation date of Mar. 4, 2016, and a size of 1 Kb. This sequence listing submitted via EFS-Web is part of the specification and is herein incorporated by reference in its entirety.

### BACKGROUND OF THE INVENTION

(1) Field of the Invention

The present invention relates to methods for identifying drug-saccharide conjugates that bind the mannose receptor, C type 1, (MRC1) and may be taken up by cells expressing MRC1, and the use of such drug-saccharide conjugates for treatment of particular diseases. In particular, the present invention relates to methods for identifying insulin-saccharide conjugates that bind the MRC1 receptor and may be taken up by cells expressing the MRC1 and use of such conjugates for treatment of diabetes.

(2) Description of Related Art

Lectins are proteins which recognize and bind specific carbohydrates, or patterns of carbohydrates (Loris, Biochimica et Biophysica Acta (BBA) 1572, 198-208 (2002)). There are many different classes of lectins with different structures and functions. Typical functions of lectins include binding to carbohydrates found on the surface of pathogens such as bacteria or yeast in order to elicit an immune response (Kilpatrick, Biochimica et Biophysica Acta (BBA) 1572, 187-197 (2002)).

Common classes of lectins found in animals include C type lectins, which depend on calcium for their binding ability, S type lectins which bind to sulfhydryl or □-galactoside groups, and P type lectins, which bind to phosphomannosyl groups (Kilpatrick, Biochimica et Biophysica Acta (BBA) 1572, 187-197 (2002)). These classes can be further subdivided based on their specific structures, binding abilities and functions. For example, C type lectins all contain a specific type of carbohydrate binding domain known as a “C type lectin-like domain”, or CTLD. However, the various subclasses of C type lectins encompass soluble and cell based receptors, molecules with single or multiple CTLDs, and have differing affinities for various sugars (Kilpatrick, Biochimica et Biophysica Acta (BBA) 1572, 187-197 (2002); East & Isacke, Biochimica et Biophysica Acta (BBA) 1572, 364-386 (2002)).

One key subclass of C type lectins are Group III C-type lectins, or collectins. Collectins are soluble proteins which have a single C type recognition domain and a collagenous domain. They are capable of forming oligomers with a higher avidity for specific carbohydrate domains than the monomeric form (Kilpatrick, Biochimica et Biophysica Acta (BBA) 1572, 187-197 (2002)). Common collectins include Mannose Binding Lectin (MBL), which can directly and indirectly activate the complement system. Surfactant Proteins-A and -D are found mainly in the lungs and bind to a variety of pathogens. Unlike MBL, SP-A and SP-D cannot directly activate the complement system; they can act as opsonins as well as cause aggregation of pathogens, altering their ability to be phagocytosed (Kerrigan & Brown, Immunobiol. 214, 562-575 (2009)).

Another key subclass of C type lectins are Group VI C type lectins, known as the mannose receptor family. This group of transmembrane lectins is defined by its multiple CTLDs, N-terminal cysteine rich domain, and fibronectin type II domain. The prototypical member of this family is the macrophage mannose receptor (MMR; MRC1), although there are several other members of the mannose receptor family, including the PLA2 receptor, DEC-205, and ENDO180 (East & Isacke, Biochimica et Biophysica Acta (BBA) 1572, 364-386 (2002)). Like the collectins, a main function of MMR is to recognize endogenous waste proteins and pathogens via their surface glycosylation. The receptors constitutively recycle between the cell surface and the interior of the cell. MMR recognizes several different patterns of carbodydrate, preferentially binding to terminal mannose, L-fucose, and N-acetylglucosamine residues, binding glucose to a lower degree, and showing little if any affinity for galactose (Taylor et al., J. Biol. Chem. 267, 1719 (1992)). MMR was first discovered on macrophages, but it is also found in some amount on other cell types, including dendritic cells, lymphatic and liver sinusoidal endothelial cells, retinal pigment epithelium, kidney mesangial cells, and tracheal smooth muscle cells (East & Isacke, Biochimica et Biophysica Acta (BBA) 1572, 364-386 (2002)).

International published application WO2010088294 discloses that certain drug conjugates, when modified to include high affinity saccharide ligands, exhibit pharmacokinetic and/or pharmacodynamics (PK/PD) profiles that responded to saccharide concentration changes, even in the absence of an exogenous multivalent saccharide-binding molecule such as Con A. This finding provides an opportunity to generate saccharide-responsive drug systems. In general, these conjugates include a drug, e.g., insulin, and one or more separate ligands that each includes a saccharide. The ligands are capable of competing with a saccharide (e.g., glucose or mannose) for binding to an endogenous saccharide-binding molecule.

### BRIEF SUMMARY OF THE INVENTION

The present invention is premised on the discovery that certain drug-saccharide conjugates, when introduced into a human subject, bind to the endogenous mannose receptor, C type 1, (MRC1). The binding is sensitive to serum concentration of glucose in the subject: competes with glucose for binding to the MRC1. At low serum levels of glucose, the drug conjugate is bound to the MRC1 and is unavailable to act at its intended site of action. For example, when the drug conjugate is an insulin conjugate, the insulin conjugate is sequestered from and unable to bind the insulin receptor. However, at elevated glucose concentrations, the glucose competes with the insulin conjugate for binding to the MRC1 and displaces insulin conjugate from the MRC1 in a concentration dependent manner. The displaced insulin conjugate is available for binding to the insulin receptor. The discovery that certain insulin-saccharide conjugates bind the MRC1 in a glucose-sensitive manner provides the foundation for the present invention.

The present invention provides a method for determining whether a drug-saccharide conjugate is capable of binding to or binds a mannose receptor, C type 1 (MRC1), comprising (a) providing MRC1 immobilized on an solid support; (b) exposing the immobilized MRC1 to a predetermined amount of a control conjugate linked to mannose and a detectable label and a predetermined amount of drug-saccharide conjugate for a time sufficient for the control conjugate and/or the drug-conjugate to bind to the MRC1; and (c) removing unbound control conjugate and drug-saccharide conjugate and measuring the amount of control conjugate bound to the MRC1, wherein a decrease in the amount of control conjugate bound to the MRC1 compared to the amount of control conjugate bound to the MRC1 in the absence of the drug-saccharide conjugate indicates the drug-saccharide conjugate is capable of binding to or binds the MRC1.

In a further embodiments, a multiplicity of MRC1 immobilized on a solid support are provided and each MRC1 immobilized on a solid support is independently exposed to the same predetermined amount of the control conjugate and a different predetermined amount of drug-saccharide conjugate for a time sufficient for the control conjugate and/or drug-conjugate to bind to the MRC1.

In further aspects, the drug is an insulin or insulin analog molecule.

In further aspects, the detectable label is a fluorescent molecule.

In further aspects, the detectable label is europium.

In further aspects, the MRC1 is a human MRC1.

In further aspects, the control conjugate comprises a protein.

In further aspects, the protein is bovine serum albumen.

The present invention provides a method for selecting a drug-saccharide conjugate that exhibits decreased uptake by target cells that express a human mannose receptor, C type 1 (MRC1) in the presence of an inhibitor from a plurality of candidate drug-saccharide congugates, comprising (a) providing the target cells that express the human MRC1; (b) exposing the target cells to the plurality of candidate drug-saccharide conjugates and selecting candidate drug-saccharide conjugates that are taken up into the target cells; (d) determining whether the uptake of the selected candidate drug-saccharide conjugate is decreased when the target cells are exposed to the selected candidate drug-saccharide conjugate and an inhibitor that binds the human MRC1; and (e) selecting at least one candidate drug-saccharide conjugate which exhibits uptake by the target cells in the absence of the inhibitor and decreased uptake in the presence of the inhibitor to select the drug-saccharide conjugate.

In further aspects, the drug is an insulin molecule.

In further aspects, the target cells are mammalian host cells that include an expression vector encoding the human MRC1 and which overexpress the human MRC1.

In further aspects, the target cells are human macrophage cells that express the MRC1.

In further aspects, the inhibitor is mannan, α-methyl mannose, or glucose.

In further aspects, the at least one candidate drug-saccharide conjugate comprise a detectable label.

In further aspects, the detectable label is fluorescent.

In further aspects, the step of determining whether the uptake of a selected candidate drug-saccharide conjugate is decreased in the presence of the inhibitor is performed at a plurality of selected candidate drug-saccharide conjugate concentrations.

In further aspects, the step of determining whether the uptake of a selected candidate drug-saccharide conjugates is decreased in the presence of the inhibitor is performed at a plurality of inhibitor concentrations.

In further aspects, the inhibitor comprises a detectable label.

In further aspects, the detectable label is fluorescent.

The present invention provides a method for selecting a drug-saccharide conjugate which decreases uptake of a control compound by target cells that express the human mannose receptor, C type 1 (MRC1), comprising (a) providing the target cells that express the human MRC1; (b) exposing the target cells to a control compound that binds the human MRC1 and is internalized into the target cells and a plurality of candidate drug-saccharide conjugates; (c) determining whether the uptake of the control compound is decreased when the target cells are exposed to a candidate drug-saccharide conjugate; and (e) selecting the candidate drug-saccharide conjugate which inhibits uptake of the control compound by the target cells in the to provide the drug-saccharide conjugate.

In further aspects, the drug is an insulin molecule.

In further aspects, the target cells are mammalian host cells that include an expression vector encoding the human MRC1 and which overexpress the human MRC1.

In further aspects, the target cells are human macrophage cells that express the MRC1.

In further aspects, the control compound comprises ovalbumin or zymosan.

In further aspects, the control compound is a drug-saccharide conjugate.

In further aspects, the control compound comprises a detectable label.

In further aspects, the detectable label is fluorescent.

In further aspects, the step of determining whether the candidate drug-saccharide conjugate decreases the uptake of the control compound is performed at a plurality of candidate drug-saccharide conjugate concentrations.

In further aspects, the step of determining whether the presence of the candidate drug-saccharide conjugate decreases the uptake of the control compound is performed at a plurality of control compound concentrations.

In any one of the aforementioned embodiments, the method may in particular aspects further include a step of determining the ability of the insulin-saccharide conjugates that bind the MRC1 or are uptaken by target cells that express the human MRC1 to also bind the insulin receptor as determined by an insulin receptor binding competition assay and/or induce insulin receptor phosphorylation as determined by an insulin receptor phosphorylation assay.

In particular aspects, the insulin receptor binding assay comprises providing target cells that express the human insulin receptor; exposing the target cells to the insulin-saccharide conjugate and insulin conjugated to a detectable label; and determining the amount of insulin-saccharide conjugate that binds to the insulin receptor. In a particular aspect, the insulin-saccharide conjugate is conjugated to a detectable label, which in particular aspects may be a fluorescent or europium detectable label.

In particular aspects, the insulin receptor binding assay comprises providing membranes that have thereon the human insulin receptor; exposing the membranes to the insulin-saccharide conjugate and insulin conjugated to a detectable label; and determining the amount of insulin-saccharide conjugate that binds to the insulin receptor. In a particular aspect, the insulin-saccharide conjugate is conjugated to a detectable label, which in particular aspects may be a fluorescent or europium detectable label.

In particular aspects, the insulin receptor phosphorylation assay comprises providing target cells that express the human insulin receptor; exposing the target cells to the insulin-saccharide conjugate; and determining the amount of phosphorylated insulin receptor.

The present invention provides a method for treating diabetes, comprising administering to a subject with diabetes a composition comprising an effective amount of an insulin-saccharide conjugate capable of binding for a mannose receptor, C type 1, (MRC1) with an half-maximal inhibitory concentration (IC₅₀) less than 20 mM.

In further aspects, the IC₅₀ is determined by one or more of the methods disclosed herein.

The present invention provides for the use of an insulin-saccharide conjugate capable of binding mannose receptor, C type 1, (MRC1) with an IC₅₀ less than 20 mM for treatment of diabetes.

In further aspects, the IC₅₀ is determined by one or more of the methods disclosed herein.

The present invention provides for the use of an insulin-saccharide conjugate capable of binding mannose receptor, C type 1, (MRC1) with an IC₅₀ less than 20 mM for the manufacture of a medicament for the treatment of diabetes.

In further aspects, the IC₅₀ is determined by one or more of the methods disclosed herein.

The present invention provides for the use of a composition comprising an insulin-saccharide conjugate capable of binding mannose receptor, C type 1, (MRC1) with an IC₅₀ less than 20 mM for treatment of diabetes.

In further aspects, the IC₅₀ is determined by one or more of the methods disclosed herein.

The present invention provides for the use of a composition comprising an insulin-saccharide conjugate capable of binding mannose receptor, C type 1, (MRC1) with an IC₅₀ less than 20 mM for the manufacture of a medicament for the treatment of diabetes.

In further aspects, the IC₅₀ is determined by one or more of the methods disclosed herein.

The present invention provides a kit, comprising:

(a) host cells transfected with an expression vector encoding a human mannose receptor, C type 1, (MRC1) and which overexpress the human MRC1; and

(b) a control compound that binds MRC1 and is internalized into the host cells.

In further aspects, the kit further includes an inhibitor selected from mannan, glucose, or alpha-methylmannose.

In further aspects, the kit further includes a drug-saccharide conjugate.

In further aspects, the control compound is ovalbumin, zymosan, or a drug-saccharide conjugate.

### Definitions

Insulin—as used herein, the term means the active principle of the pancreas that affects the metabolism of carbohydrates in the animal body and which is of value in the treatment of diabetes mellitus. The term includes synthetic and biotechnologically derived products that are the same as, or similar to, naturally occurring insulins in structure, use, and intended effect and are of value in the treatment of diabetes mellitus.

Insulin or insulin molecule—the term is a generic term that designates the 51 amino acid heterodimer comprising the A-chain peptide having the amino acid sequence shown in SEQ ID NO: 1 and the B-chain peptide having the amino acid sequence shown in SEQ ID NO: 2, wherein the cysteine residues a positions 6 and 11 of the A chain are linked in a disulfide bond, the cysteine residues at position 7 of the A chain and position 7 of the B chain are linked in a disulfide bond, and the cysteine residues at position 20 of the A chain and 19 of the B chain are linked in a disulfide bond.

Insulin analog or analog—the term as used herein includes any heterodimer analogue or single-chain analogue that comprises one or more modification(s) of the native A-chain peptide and/or B-chain peptide. Modifications include but are not limited to substituting an amino acid for the native amino acid at a position selected from A4, A5, A8, A9, A10, A12, A13, A14, A15, A16, A17, A18, A19, A21, B1, B2, B3, B4, B5, B9, B10, B13, B14, B15, B16, B17, B18, B20, B21, B22, B23, B26, B27, B28, B29, and B30; deleting any or all of positions B1-4 and B26-30; or conjugating directly or by a polymeric or non-polymeric linker one or more acyl, polyethylglycine (PEG), or saccharide moiety (moieties); or any combination thereof. As exemplified by the N-linked glycosylated insulin analogues disclosed herein, the term further includes any insulin heterodimer and single-chain analogue that has been modified to have at least one N-linked glycosylation site and in particular, embodiments in which the N-linked glycosylation site is linked to or occupied by an N-glycan. Examples of insulin analogues include but are not limited to the heterodimer and single-chain analogues disclosed in published international application WO20100080606, WO2009/099763, and WO2010080609, the disclosures of which are incorporated herein by reference. Examples of single-chain insulin analogues also include but are not limited to those disclosed in published International Applications WO9634882, WO95516708, WO2005054291, WO2006097521, WO2007104734, WO2007104736, WO2007104737, WO2007104738, WO2007096332, WO2009132129; U.S. Pat. Nos. 5,304,473 and 6,630,348; and Kristensen et al., Biochem. J. 305: 981-986 (1995), the disclosures of which are each incorporated herein by reference.

The term further includes single-chain and heterodimer polypeptide molecules that have little or no detectable activity at the insulin receptor but which have been modified to include one or more amino acid modifications or substitutions to have an activity at the insulin receptor that has at least 1%, 10%, 50%, 75%, or 90% of the activity at the insulin receptor as compared to native insulin and which further includes at least one N-linked glycosylation site. In particular aspects, the insulin analogue is a partial agonist that has from 2× to 100× less activity at the insulin receptor as does native insulin. In other aspects, the insulin analogue has enhanced activity at the insulin receptor, for example, the IGF^{B16B17} derivative peptides disclosed in published international application WO2010080607 (which is incorporated herein by reference). These insulin analogues, which have reduced activity at the insulin growth hormone receptor and enhanced activity at the insulin receptor, include both heterodimers and single-chain analogues.

Single-chain insulin or single-chain insulin analog—as used herein, the term encompasses a group of structurally-related proteins wherein the A-chain peptide or functional analogue and the B-chain peptide or functional analogue are covalently linked by a peptide or polypeptide of 2 to 35 amino acids or non-peptide polymeric or non-polymeric linker and which has at least 1%, 10%, 50%, 75%, or 90% of the activity of insulin at the insulin receptor as compared to native insulin. The single-chain insulin or insulin analogue further includes three disulfide bonds: the first disulfide bond is between the cysteine residues at positions 6 and 11 of the A-chain or functional analogue thereof, the second disulfide bond is between the cysteine residues at position 7 of the A-chain or functional analogue thereof and position 7 of the B-chain or functional analogue thereof, and the third disulfide bond is between the cysteine residues at position 20 of the A-chain or functional analogue thereof and position 19 of the B-chain or functional analogue thereof.

Drug—As used herein, the term “drug” refers to small molecules or biomolecules that alter, inhibit, activate, or otherwise affect a biological event. For example, drugs may include, but are not limited to, anti-AIDS substances, anti-cancer substances, antibiotics, anti-diabetic substances, immunosuppressants, anti-viral substances, enzyme inhibitors, neurotoxins, opioids, hypnotics, anti-histamines, lubricants, tranquilizers, anti-convulsants, muscle relaxants and anti-Parkinson substances, anti-spasmodics and muscle contractants including channel blockers, miotics and anti-cholinergics, anti-glaucoma compounds, anti-parasite and/or anti-protozoal compounds, modulators of cell-extracellular matrix interactions including cell growth inhibitors and anti-adhesion molecules, vasodilating agents, inhibitors of DNA, RNA or protein synthesis, anti-hypertensives, analgesics, anti-pyretics, steroidal and non-steroidal anti-inflammatory agents, anti-angiogenic factors, anti-secretory factors, anticoagulants and/or anti-thrombotic agents, local anesthetics, ophthalmics, prostaglandins, anti-depressants, anti-psychotic substances, anti-emetics, and imaging agents.

Treat—As used herein, the term “treat” (or “treating”, “treated”, “treatment”, etc.) refers to the administration of a conjugate of the present disclosure to a subject in need thereof with the purpose to alleviate, relieve, alter, ameliorate, improve or affect a condition (e.g., diabetes), a symptom or symptoms of a condition (e.g., hyperglycemia), or the predisposition toward a condition.

### BRIEF DESCRIPTION OF THE DRAWINGS

FIG. 1A shows a Western blot

FIG. 1B shows uptake of Compound I by rat NR8383 cells is reduced when expression of MRC1 has been reduced (knocked down) by anti-MRC1 siRNA.

FIG. 2A shows MRC1 knockdown in human macrophage cells treated by anti-MRC1 siRNA.

FIG. 2B shows uptake of Compound I is reduced in human macrophage cells when expression of MRC1 has been reduced (knocked down) by anti-MRC1 siRNA.

FIG. 3 shows that over expression of MRC1 in HEK293 cells increased Compound I uptake by the cells.

FIG. 4A shows a cartoon of a competition assay for identifying molecules that can bind the MRC1 and can measure the potency of the binding.

FIG. 4B shows that Compound I binds to MRC1 competitively

FIGS. 5A and 5B shows that MRC1 has a predominant role in clearance of insulin-saccharide conjugates such as Compound II (5B) compared to insulin (5A) in mice.

FIG. 6A shows the formula for insulin-saccharide conjugate Compound I (TSAT-C6-Di-sub-AETM-2 (A1,B29)).

FIG. 6B shows the formula for insulin-saccharide conjugate Compound II (TSAT-C6-AETM-2 (B29)).

### DETAILED DESCRIPTION OF THE INVENTION

Recently, it was shown that when certain saccharides are conjugated to a drug (e.g., an insulin molecule) and administered to a subject (e.g., a rat, a mini-pig, etc.), the resulting insulin-saccharide conjugate exhibits pharmacokinetic (PK) and pharmacodynamic (PD) properties that vary with systemic glucose concentration (e.g., see WO 2010/88294 which is incorporated herein by reference in its entirety). In particular, classes of drug-saccharide conjugates that exhibit longer lifetimes under hyperglycemic conditions than under hypoglycemic conditions have been identified. In light of these properties, these “glucose-responsive” drug-saccharide conjugates have a greater effect on the patient when glucose concentrations are high than when they are low. This is particularly useful when the drug is an insulin molecule since insulin is only needed by the subject under hyperglycemic conditions and would in fact have a negative impact if it exerted an effect under hypoglycemic conditions. However, as discussed in WO 2010/88294, the conjugates are also useful for drugs other than insulin.

In WO2010/88294, it was postulated that the “glucose-responsive” nature of these conjugates may result from binding between the saccharide components of the conjugates and one or more endogenous lectins in the subject. In WO2012050822, a method for identifying such conjugates by measuring uptake of the conjugate into NR8383 rat alveolar macrophage cells was described.

The present disclosure describes experiments that have allowed us for the first time to identify that in human subjects, certain classes of drug-saccharide conjugates will bind the human mannose receptor, C type 1 (MRC1) and that the interaction is sensitive to the serum concentration of glucose or an endogenously administered saccharide such as alpha-methylmannose. Having identified the MRC1 as the relevant endogenous lectin for binding drug-saccharide conjugates, we can now use the MRC1 in assays to screen other compounds (not necessarily saccharides) for their ability to bind with MRC1 and be uptake by cells displaying the MRC1. In particular we can now screen different compounds for their binding affinities with MRC1 and also assess how this binding is affected by different concentrations of glucose.

Based on our results, we can now also select compounds that are known to bind the MRC1 (e.g., based on previous studies) and include these in an inventive conjugate. The present disclosure encompasses these other compounds and their use as components of inventive conjugates. We can also use this information to identify other compounds (again not necessarily saccharides) that can inhibit the binding between previously identified conjugates (or their saccharide components) and MRC1. These other compounds may be useful as modulators of the interactions between a drug-saccharide conjugate and MRC1. The present disclosure encompasses these other compounds and their uses as modulators of inventive conjugates. The present disclosure also encompasses these screening methods and associated compositions of matter, e.g., kits, cell lines, etc. that can be used to perform the screening methods.

### Conjugates

In general, the conjugates that are tested in the methods disclosed herein include at least one ligand. In certain embodiments, the conjugates include a single ligand. In certain embodiments, the conjugates include at least two separate ligands, e.g., 2, 3, 4, 5 or more ligands. When more than one ligand is present, the ligands may have the same or different chemical structures. Examples of ligands are disclosed in WO2010088294, which is incorporated herein in its entirety.

In certain embodiments, the ligand is of formula (IIIa) or (IIIb):

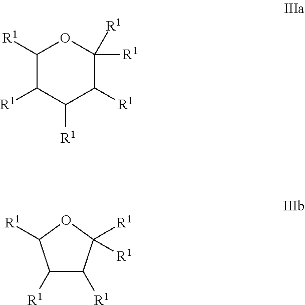

wherein:
each R¹ is independently hydrogen, —OR^{y}, —N(R^{y})₂, —SR^{y}, —O—Y, -G-Z, or —CH₂R^{x};
each R^{x} is independently hydrogen, —OR^{y}, —N(R^{y})₂, —SR^{y}, or —O—Y;
each R^{y} is independently —R², —SO₂R², —S(O)R², —P(O)(OR²)₂, —C(O)R², —CO₂R², or —C(O)N(R²)₂;
each Y is independently a monosaccharide, disaccharide, or trisaccharide;
each G is independently a covalent bond or an optionally substituted C₁₋₉ alkylene, wherein one or more methylene units of G is optionally replaced by —O—, —S—, —N(R²)—, —C(O)—, —OC(O)—, —C(O)O—, —C(O)N(R²)—, —N(R²)C(O)—, —N(R²)C(O)N(R²)—, —SO₂—, —SO₂N(R²)—, —N(R²)SO₂—, or —N(R²)SO₂N(R²)—;
each Z is independently halogen, —N(R²)₂, —OR², —SR², —N₃, —C≡CR², —CO₂R², —C(O)R², or —OSO₂R²; and
each R² is independently hydrogen or an optionally substituted group selected from C₁₋₆ aliphatic, phenyl, a 4-7 membered heterocyclic ring having 1-2 heteroatoms selected from nitrogen, oxygen, or sulfur, or a 5-6 membered monocyclic heteroaryl ring having 1-4 heteroatoms selected from nitrogen, oxygen, or sulfur.

In certain embodiments, the ligand of formula (IIIa) or (IIIb) is a monosaccharide. In certain embodiments, the ligand is a disaccharide. In certain embodiments, the ligand is a trisaccharide. In certain embodiments, the ligand is a tetrasaccharide. In certain embodiments, the ligand comprises no more than a total of four monosaccharide moieties.

As defined generally above, each R¹ is independently hydrogen, —OR^{y}, —N(R^{y})₂, —SR^{y}, —O—Y, -G-Z, or —CH₂R^{x}. In certain embodiments, R¹ is hydrogen. In certain embodiments, R¹ is —OH. In other embodiments, R¹ is —NHC(O)CH₃. In certain embodiments, R¹ is —O—Y. In certain other embodiments, R¹ is -G-Z. In some embodiments, R¹ is —CH₂OH. In other embodiments, R¹ is —CH₂—O—Y. In yet other embodiments, R¹ is —NH₂. One of ordinary skill in the art will appreciate that each R¹ substituent in formula (IIIa) or (IIIb) may be of (R) or (S) stereochemistry.

As defined generally above, each R^{x} is independently hydrogen, —OR^{y}, —N(R^{y})₂, —SR^{y}, or —O—Y. In some embodiments, R^{x} is hydrogen. In certain embodiments, R^{x} is —OH. In other embodiments, R^{x} is —O—Y.

As defined generally above, each R^{y} is independently —R², —SO₂R², —S(O)R², —P(O)(OR²)₂, —C(O)R², —CO₂R², or —C(O)N(R²)₂. In some embodiments, R^{y} is hydrogen. In other embodiments, R^{y} is —R². In some embodiments, R^{y} is —C(O)R². In certain embodiments, R^{y} is acetyl. In other embodiments, R^{y} is —SO₂R², —S(O)R², —P(O)(OR²)₂, —CO₂R², or —C(O)N(R²)₂.

As defined generally above, Y is a monosaccharide, disaccharide, or trisaccharide. In certain embodiments, Y is a monosaccharide. In some embodiments, Y is a disaccharide. In other embodiments, Y is a trisaccharide. In some embodiments, Y is mannose, glucose, fructose, galactose, rhamnose, or xylopyranose. In some embodiments, Y is sucrose, maltose, turanose, trehalose, cellobiose, or lactose. In certain embodiments, Y is mannose. In certain embodiments, Y is D-mannose. One of ordinary skill in the art will appreciate that the saccharide Y is attached to the oxygen group of —O—Y through anomeric carbon to form a glycosidic bond. The glycosidic bond may be of an alpha or beta configuration.

As defined generally above, each G is independently a covalent bond or an optionally substituted C₁₋₉ alkylene, wherein one or more methylene units of G is optionally replaced by —O—, —S—, —N(R²)—, —C(O)—, —OC(O)—, —C(O)O—, —C(O)N(R²)—, —N(R²)C(O)—, —N(R²)C(O)N(R²)—, —SO₂—, —SO₂N(R²)—, —N(R²)SO₂—, or —N(R²)SO₂N(R²)—. In some embodiments, G is a covalent bond. In certain embodiments, G is —O—C₁₋₈ alkylene. In certain embodiments, G is —OCH₂CH₂—.

As defined generally above, each Z is independently halogen, —N(R²)₂, —OR², —SR², —N₃, —C≡CR², —CO₂R², —C(O)R², or —OSO₂R². In some embodiments, Z is a halogen or —OSO₂R². In other embodiments, Z is —N₃ or —C≡CR². In certain embodiments, Z is —N(R²)₂, —OR², or —SR². In certain embodiments, Z is —SH. In certain embodiments, Z is —NH₂. In certain embodiments, -G-Z is —OCH₂CH₂NH₂.

In some embodiments, the R¹ substituent on the C1 carbon of formula (IIIa) is -G-Z to give a compound of formula (IIIa-i):

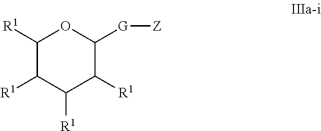

wherein R¹, G, and Z are as defined and described herein.

In some embodiments, the ligand is of formula (IIIa-ii):

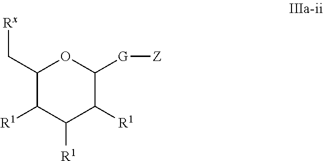

wherein R¹, R^{x}, G, and Z are as defined and described herein.

In certain embodiments, the ligand(s) may have the same chemical structure as glucose or may be a chemically related species of glucose. In various embodiments it may be advantageous for the ligand(s) to have a different chemical structure from glucose, e.g., in order to fine tune the glucose response of the conjugate. For example, in certain embodiments, one might use a ligand that includes glucose, mannose, L-fucose or derivatives of these (e.g., alpha-L-fucopyranoside, mannosamine, beta-linked N-acetyl mannosamine, methylglucose, methylmannose, ethylglucose, ethylmannose, propylglucose, propylmannose, etc.) and/or higher order combinations of these (e.g., a bimannose, linear and/or branched trimannose, etc.).

In certain embodiments, the ligand includes a monosaccharide. In certain embodiments, the ligand includes a disaccharide. In certain embodiments, the ligand is includes a trisaccharide. In some embodiments, the ligand comprises a saccharide and one or more amine groups. In certain embodiments the saccharide and amine group are separated by a C₁-C₆ alkyl group, e.g., a C₁-C₃ alkyl group. In some embodiments, the ligand is aminoethylglucose (AEG). In some embodiments, the ligand is aminoethylmannose (AEM). In some embodiments, the ligand is aminoethylbimannose (AEBM). In some embodiments, the ligand is aminoethyltrimannose (AETM). In some embodiments, the ligand is β-aminoethyl-N-acetylglucosamine (AEGA). In some embodiments, the ligand is aminoethylfucose (AEF). In certain embodiments, a saccharide ligand is of the “D” configuration. In other embodiments, a saccharide ligand is of the “L” configuration. Below we show the structures of these exemplary ligands. Other exemplary ligands will be recognized by those skilled in the art.

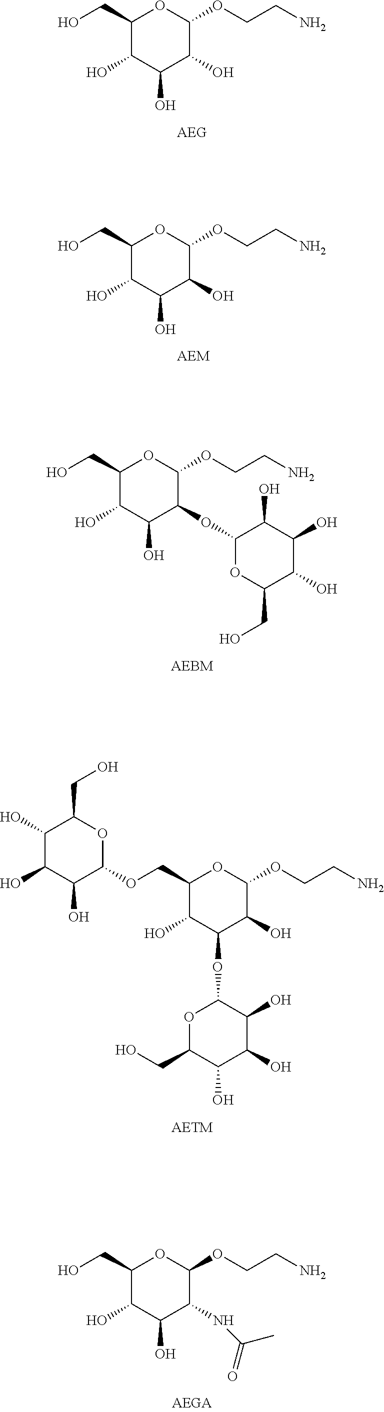 [SMILES: NCCO[C@H]1O[C@H](CO)[C@@H](O)[C@H](O)[C@H]1O ; NCCO[C@H]1O[C@H](CO)[C@@H](O)[C@H](O)[C@@H]1O ; NCCO[C@H]1O[C@H](CO)[C@@H](O)[C@H](O)[C@@H]1O[C@H]1O[C@H](CO)[C@@H](O)[C@H](O)[C@@H]1O ; NCCO[C@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O ; CC(=O)N[C@H]1[C@H](OCCN)O[C@H](CO)[C@@H](O)[C@@H]1O]

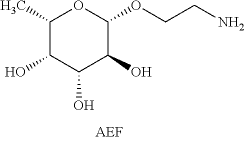

In general, ligands may be directly or indirectly conjugated (i.e., via a linker or framework) to the drug. As discussed in more detail below, the ligands may be naturally present within a conjugate framework (e.g., as part of a polymer backbone or as a side group of a monomer). Alternatively (or additionally) ligands may be artificially incorporated into a conjugate framework (e.g., in the form of a chemical group that is synthetically added to a conjugate framework). In certain embodiments, a conjugate may include a framework which comprises 5 or more, 10 or more, or 20 or more ligands. In certain embodiments, a conjugate may comprise as few as 1, 2, 3, 4 or 5 separate ligands.

In certain embodiments, at least two separate ligands are conjugated to the drug via different conjugation points. In certain embodiments, at least two separate ligands are conjugated to a single conjugate framework that is also conjugated to the drug. In some embodiments, at least one ligand, such as AETM, AEG, AEM, AEBM, AEGA, or AEF, is conjugated to one insulin molecule. In certain embodiments, at least one AETM ligand is conjugated to one insulin molecule. In some embodiments, at least two ligands, such as AETM, AEG, AEM, AEBM, AEGA, or AEF, are conjugated to one insulin molecule, either through one conjugation point or multiple conjugation points. In certain embodiments, the at least two ligands are not the same ligand. In certain embodiments, the at least two ligands are the same ligand. In certain embodiments, at least two AETM ligands are conjugated to one insulin molecule, either through one conjugation point or multiple conjugation points. As discussed in more detail below in the context of certain exemplary conjugate frameworks, in certain embodiments the separate ligands and drug (e.g., an insulin molecule) may each be located on a separate branch of a branched conjugate framework. For example, the ligands and drug may be located on termini of these branches. In certain embodiments a hyperbranched conjugate framework may be used. Both polymeric and non-polymeric conjugate frameworks are encompassed.

Methods for conjugating ligands to a conjugate framework are discussed in more detail below. In certain embodiments, the saccharide within the one or more ligands is conjugated (directly or indirectly by way of a linker) via the C1, C2 or C6 position. In certain embodiments, the conjugation involves the C1 position. The C1 position of a saccharide is also referred to as the anomeric carbon and may be connected to the drug or conjugate framework in the alpha or beta conformation. In certain embodiments, the C1 position is configured as the alpha anomer. In other embodiments, the C1 position is configured as the beta anomer.

### Drug

It is to be understood that a conjugate can comprise any drug. A conjugate can comprise more than one copy of the same drug and/or can comprise more than one type of drug.

The conjugates are not limited to any particular drug and may include small molecule drugs or biomolecular drugs. In general, the drug(s) used will depend on the disease or disorder to be treated. As used herein, the term “drug” encompasses salt and non-salt forms of the drug. For example, the term “insulin molecule” encompasses all salt and non-salt forms of the insulin molecule. It will be appreciated that the salt form may be anionic or cationic depending on the drug.

Examples of drugs that may comprise the conjugate are disclosed in WO2010088294, which is incorporated herein in its entirety. For example, without limitation, in various embodiments a conjugate may comprise any one of the following drugs: diclofenac, nifedipine, rivastigmine, methylphenidate, fluoroxetine, rosiglitazone, prednison, prednisolone, codeine, ethylmorphine, dextromethorphan, noscapine, pentoxiverine, acetylcysteine, bromhexine, epinephrine, isoprenaline, orciprenaline, ephedrine, fenoterol, rimiterol, ipratropium, cholinetheophyllinate, proxiphylline, bechlomethasone, budesonide, deslanoside, digoxine, digitoxin, disopyramide, proscillaridin, chinidine, procainamide, mexiletin, flecainide, alprenolol, proproanolol, nadolol, pindolol, oxprenolol, labetalol, tirnolol, atenolol, pentaeritrityltetranitrate, isosorbiddinitrate, isosorbidmononitrate, niphedipin, phenylamine, verapamil, diltiazem, cyclandelar, nicotinylalcholhol, inositolnicotinate, alprostatdil, etilephrine, prenalterol, dobutamine, dopamine, dihydroergotamine, guanetidine, betanidine, methyldopa, reserpine, guanfacine, trimethaphan, hydralazine, dihydralazine, prazosine, diazoxid, captopril, nifedipine, enalapril, nitroprusside, bendroflumethiazide, hydrochlorthiazide, metychlothiazide, polythiazide, chlorthalidon, cinetazon, clopamide, mefruside, metholazone, bumetanide, ethacrynacide, spironolactone, amiloride, chlofibrate, nicotinic acid, nicheritrol, brompheniramine, cinnarizine, dexchlorpheniramine, clemastine, antazoline, cyproheptadine, proethazine, cimetidine, ranitidine, sucralfat, papaverine, moxaverine, atropin, butylscopolamin, emepron, glucopyrron, hyoscyamine, mepensolar, methylscopolamine, oxiphencyclimine, probanteline, terodilin, sennaglycosides, sagradaextract, dantron, bisachodyl, sodiumpicosulfat, etulos, diphenolxylate, loperamide, salazosulfapyridine, pyrvin, mebendazol, dimeticon, ferrofumarate, ferrosuccinate, ferritetrasemisodium, cyanochobalamine, folid acid heparin, heparin co-factor, diculmarole, warfarin, streptokinase, urokinase, factor VIII, factor IX, vitamin K, thiopeta, busulfan, chlorambucil, cyclophosphamid, melfalan, carmustin, mercatopurin, thioguanin, azathioprin, cytarabin, vinblastin, vinchristin, vindesin, procarbazine, dacarbazine, lomustin, estramustin, teniposide, etoposide, cisplatin, amsachrin, aminogluthetimid, phosphestrol, medroxiprogresterone, hydroxiprogesterone, megesterol, noretisteron, tamoxiphen, ciclosporin, sulfosomidine, bensylpenicillin, phenoxymethylpenicillin, dicloxacillin, cloxacillin, flucoxacillin, ampicillin, amoxicillin, pivampicillin, bacampicillin, piperacillin, meziocillin, mecillinam, pivmecillinam, cephalotin, cephalexin, cephradin, cephadroxil, cephaclor, cefuroxim, cefotaxim, ceftazidim, cefoxitin, aztreonam, imipenem, cilastatin, tetracycline, lymecycline, demeclocycline, metacycline, oxitetracycline, doxycycline, chloramphenicol, spiramycin, fusidic acid, lincomycin, clindamycin, spectinomycin, rifampicin, amphotericin B, griseofulvin, nystatin, vancomycin, metronidazole, tinidazole, trimethoprim, norfloxacin, salazosulfapyridin, aminosalyl, isoniazid, etambutol, nitrofurantoin, nalidixic acid, metanamine, chloroquin, hydroxichloroquin, tinidazol, ketokonazol, acyclovir, interferon idoxuridin, retinal, tiamin, dexpantenol, pyridoxin, folic acid, ascorbic acid, tokoferol, phytominadion, phenfluramin, corticotropin, tetracosactid, tyrotropin, somatotoprin, somatrem, vasopressin, lypressin, desmopressin, oxytocin, chloriongonadotropin, cortison, hydrocortisone, fluodrocortison, prednison, prednisolon, fluoximesteron, mesterolon, nandrolon, stanozolol, oximetolon, cyproteron, levotyroxin, liotyronin, propylthiouracil, carbimazol, tiamazol, dihydrotachysterol, alfacalcidol, calcitirol, insulin, tolbutamid, chlorpropamid, tolazamid, glipizid, glibenclamid, phenobarbital, methyprylon, pyrityidion, meprobamat, chlordiazepoxid, diazepam, nitrazepam, baclofen, oxazepam, dikaliumclorazepat, lorazepam, flunitrazepam, alprazolam, midazolam, hydroxizin, dantrolene, chlometiazol, propionmazine, alimemazine, chlorpromazine, levomepromazine, acetophenazine, fluphenazine, perphenazine, prochlorperazine, trifluoperazine, dixyrazine, thiodirazine, periciazin, chloprothixene, tizanidine, zaleplon, zuclopentizol, flupentizol, thithixen, haloperidol, trimipramin, opipramol, chlomipramin, desipramin, lofepramin, amitriptylin, nortriptylin, protriptylin, maptrotilin, caffeine, cinnarizine, cyclizine, dimenhydinate, meclozine, prometazine, thiethylperazine, metoclopramide, scopolamine, phenobarbital, phenytoine, ethosuximide, primidone, carbamazepine, chlonazepam, orphenadrine, atropine, bensatropine, biperiden, metixene, procylidine, levodopa, bromocriptin, amantadine, ambenon, pyridostigmine, synstigmine, disulfiram, morphine, codeine, pentazocine, buprenorphine, pethidine, phenoperidine, phentanyl, methadone, piritramide, dextropropoxyphene, ketobemidone, acetylsalicylic acid, celecoxib, phenazone, phenylbutazone, azapropazone, piroxicam, ergotamine, dihydroergotamine, cyproheptadine, pizitifen, flumedroxon, allopurinol, probenecid, sodiummaurothiomalate auronofin, penicillamine, estradiol, estradiolvalerianate, estriol, ethinylestradiol, dihydrogesteron, lynestrenol, medroxiprogresterone, noretisterone, cyclophenile, clomiphene, levonorgestrel, mestranol, ornidazol, tinidazol, ekonazol, chlotrimazol, natamycine, miconazole, sulbentin, methylergotamine, dinoprost, dinoproston, gemeprost, bromocriptine, phenylpropanolamine, sodiumchromoglicate, azetasolamide, dichlophenamide, betacarotene, naloxone, calciumfolinate, in particular clonidine, thephylline, dipyradamol, hydrochlothiazade, scopolamine, indomethacine, furosemide, potassium chloride, morphine, ibuprofen, salbutamol, terbutalin, calcitonin, etc. It is to be understood that this list is intended to be exemplary and that any drug, whether known or later discovered, may be used in a conjugate of the present disclosure.

In various embodiments, a conjugate may include a hormonal drug which may be peptidic or non-peptidic, e.g., adrenaline, noradrenaline, angiotensin, atriopeptin, aldosterone, dehydroepiandrosterone, androstenedione, testosterone, dihydrotestosterone, calcitonin, calcitriol, calcidiol, corticotropin, cortisol, dopamine, estradiol, estrone, estriol, erythropoietin, follicle-stimulating hormone, gastrin, ghrelin, glucagon, gonadotropin-releasing hormone, growth hormone, growth hormone-releasing hormone, human chorionic gonadotropin, histamine, human placental lactogen, insulin, insulin-like growth factor, inhibin, leptin, a leukotriene, lipotropin, melatonin, orexin, oxytocin, parathyroid hormone, progesterone, prolactin, prolactin-releasing hormone, a prostglandin, renin, serotonin, secretin, somatostatin, thrombopoietin, thyroid-stimulating hormone, thyrotropin-releasing hormone (or thyrotropin), thyrotropin-releasing hormone, thyroxine, triiodothyronine, vasopressin, etc. In certain embodiments, the hormone may be selected from glucagon, glucagon-like peptide 1 (GLP-1), oxyntomodulin, insulin, insulin-like growth factor, leptin, thyroid-stimulating hormone, thyrotropin-releasing hormone (or thyrotropin), thyrotropin-releasing hormone, thyroxine, and triiodothyronine. In certain embodiments, the drug is insulin-like growth factor 1 (IGF-1). It is to be understood that this list is intended to be exemplary and that any hormonal drug, whether known or later discovered, may be used in a conjugate of the present disclosure.

In various embodiments, a conjugate may include a thyroid hormone.

In various embodiments, a conjugate may include an anti-diabetic drug (i.e., a drug which has a beneficial effect on patients suffering from diabetes).

In various embodiments, a conjugate may include an insulin molecule. As used herein, the term “insulin” or “insulin molecule” encompasses all salt and non-salt forms of the insulin molecule. It will be appreciated that the salt form may be anionic or cationic depending on the insulin molecule. By “insulin” or “an insulin molecule” we intend to encompass both wild-type and modified forms of insulin as long as they are bioactive (i.e., capable of causing a detectable reduction in glucose when administered in vivo). Wild-type insulin includes insulin from any species whether in purified, synthetic or recombinant form (e.g., human insulin, porcine insulin, bovine insulin, rabbit insulin, sheep insulin, etc.). A number of these are available commercially, e.g., from Sigma-Aldrich (St. Louis, Mo.). A variety of modified forms of insulin are known in the art (e.g. see Crotty and Reynolds, Pediatr. Emerg. Care. 23:903-905, 2007 and Gerich, Am. J. Med. 113:308-16, 2002 and references cited therein). Modified forms of insulin may be chemically modified (e.g., by addition of a chemical moiety such as a PEG group or a fatty acyl chain as described below) and/or mutated (i.e., by addition, deletion or substitution of one or more amino acids).

In certain embodiments, an insulin molecule of the present disclosure will differ from a wild-type insulin by 1-10 (e.g., 1-9, 1-8, 1-7, 1-6, 1-5, 1-4, 1-3, 1-2, 2-9, 2-8, 2-7, 2-6, 2-5, 2-4, 2-3, 3-9, 3-8, 3-7, 3-6, 3-5, 3-4, 4-9, 4-8, 4-7, 4-6, 4-5, 5-9, 5-8, 5-7, 5-6, 6-9, 6-8, 6-7, 7-9, 7-8, 8-9, 9, 8, 7, 6, 5, 4, 3, 2 or 1) amino acid substitutions, additions and/or deletions. In certain embodiments, an insulin molecule of the present disclosure will differ from a wild-type insulin by amino acid substitutions only. In certain embodiments, an insulin molecule of the present disclosure will differ from a wild-type insulin by amino acid additions only. In certain embodiments, an insulin molecule of the present disclosure will differ from a wild-type insulin by both amino acid substitutions and additions. In certain embodiments, an insulin molecule of the present disclosure will differ from a wild-type insulin by both amino acid substitutions and deletions.

In certain embodiments, amino acid substitutions may be made on the basis of similarity in polarity, charge, solubility, hydrophobicity, hydrophilicity, and/or the amphipathic nature of the residues involved. In certain embodiments, a substitution may be conservative, that is, one amino acid is replaced with one of similar shape and charge. Conservative substitutions are well known in the art and typically include substitutions within the following groups: glycine, alanine; valine, isoleucine, leucine; aspartic acid, glutamic acid; asparagine, glutamine; serine, threonine; lysine, arginine; and tyrosine, phenylalanine. In certain embodiments, the hydrophobic index of amino acids may be considered in choosing suitable mutations. The importance of the hydrophobic amino acid index in conferring interactive biological function on a polypeptide is generally understood in the art. Alternatively, the substitution of like amino acids can be made effectively on the basis of hydrophilicity. The importance of hydrophilicity in conferring interactive biological function of a polypeptide is generally understood in the art. The use of the hydrophobic index or hydrophilicity in designing polypeptides is further discussed in U.S. Pat. No. 5,691,198.

The wild-type sequence of human insulin (A-chain and B-chain) is shown below.

A-Chain (SEQ ID NO: 1):
GIVEQCCTSICSLYQLENYCN
B-Chain (SEQ ID NO: 2):
FVNQHLCGSHLVEALYLVCGERGFFYTPKT

In various embodiments, an insulin molecule of the present disclosure is mutated at the B28 and/or B29 positions of the B-peptide sequence. For example, insulin lispro (HUMALOG) is a rapid acting insulin mutant in which the penultimate lysine and proline residues on the C-terminal end of the B-peptide have been reversed (Lys^{B28}pro^{B29}-human insulin). This modification blocks the formation of insulin multimers. Insulin aspart (NOVOLOG) is another rapid acting insulin mutant in which proline at position B28 has been substituted with aspartic acid (Asp^{B28}-human insulin). This mutant also prevents the formation of multimers. In some embodiments, mutation at positions B28 and/or B29 is accompanied by one or more mutations elsewhere in the insulin polypeptide. For example, insulin glulisine (APIDRA) is yet another rapid acting insulin mutant in which aspartic acid at position B3 has been replaced by a lysine residue and lysine at position B29 has been replaced with a glutamic acid residue (Lys^{B3}Glu^{B29}-human insulin).

In various embodiments, an insulin molecule of the present disclosure has an isoelectric point that is shifted relative to human insulin. In some embodiments, the shift in isoelectric point is achieved by adding one or more arginine residues to the N-terminus of the insulin A-peptide and/or the C-terminus of the insulin B-peptide. Examples of such insulin polypeptides include Arg^{A0}-human insulin, Arg^{B31}Arg^{B32}-human insulin, Gly^{A21}Arg^{B31}Arg^{B32}-human insulin, Arg^{A0}Arg^{B31}Arg^{B32}-human insulin, and Arg^{A0}Gly^{A21}Arg^{B31}Arg^{B32}-human insulin. By way of further example, insulin glargine (LANTUS) is an exemplary long acting insulin mutant in which Asp^{A21} has been replaced by glycine, and two arginine residues have been added to the C-terminus of the B-peptide. The effect of these changes is to shift the isoelectric point, producing a solution that is completely soluble at pH 4. Thus, in some embodiments, an insulin molecule of the present disclosure comprises an A-peptide sequence wherein A21 is Gly and B-peptide sequence wherein B31 and B32 are Arg-Arg. It is to be understood that the present disclosure encompasses all single and multiple combinations of these mutations and any other mutations that are described herein (e.g., Gly^{A21}-human insulin, Gly^{A21}Arg^{B31}-human insulin, Arg^{B31}Arg^{B32}-human insulin, Arg^{B31}-human insulin).

In various embodiments, an insulin molecule of the present disclosure is truncated. For example, in certain embodiments, a B-peptide sequence of an insulin polypeptide of the present disclosure is missing B1, B2, B3, B26, B27, B28, B29 and/or B30. In certain embodiments, combinations of residues are missing from the B-peptide sequence of an insulin polypeptide of the present disclosure. For example, the B-peptide sequence may be missing residues B(1-2), B(1-3), B(29-30), B(28-30), B(27-30) and/or B(26-30). In some embodiments, these deletions and/or truncations apply to any of the aforementioned insulin molecules (e.g., without limitation to produce des(B30)-insulin lispro, des(B30)-insulin aspart, des(B30)-insulin glulisine, des(B30)-insulin glargine, etc.).

In some embodiments, an insulin molecule contains additional amino acid residues on the N- or C-terminus of the A or B-peptide sequences. In some embodiments, one or more amino acid residues are located at positions A0, A21, B0 and/or B31. In some embodiments, one or more amino acid residues are located at position A0. In some embodiments, one or more amino acid residues are located at position A21. In some embodiments, one or more amino acid residues are located at position B0. In some embodiments, one or more amino acid residues are located at position B31. In certain embodiments, an insulin molecule does not include any additional amino acid residues at positions A0, A21, B0 or B31.

In certain embodiments, an insulin molecule of the present disclosure is mutated such that one or more amidated amino acids are replaced with acidic forms. For example, asparagine may be replaced with aspartic acid or glutamic acid. Likewise, glutamine may be replaced with aspartic acid or glutamic acid. In particular, Asn^{A18}, Asn^{A21}, or Asn^{B3}, or any combination of those residues, may be replaced by aspartic acid or glutamic acid. Gln^{A15} or Gln^{B4}, or both, may be replaced by aspartic acid or glutamic acid. In certain embodiments, an insulin molecule has aspartic acid at position A21 or aspartic acid at position B3, or both.

One skilled in the art will recognize that it is possible to mutate yet other amino acids in the insulin molecule while retaining biological activity. For example, without limitation, the following modifications are also widely accepted in the art: replacement of the histidine residue of position B10 with aspartic acid (His^{B10}→Asp^{B10}); replacement of the phenylalanine residue at position B1 with aspartic acid (Phe^{B1}→Asp^{B1}); replacement of the threonine residue at position B30 with alanine (Thr^{B30}→Ala^{B30}); replacement of the tyrosine residue at position B26 with alanine (Tyr^{B26}→Ala^{B26}); and replacement of the serine residue at position B9 with aspartic acid (Ser^{B9}→Asp^{B9}).

In various embodiments, an insulin molecule of the present disclosure has a protracted profile of action. Thus, in certain embodiments, an insulin molecule of the present disclosure may be acylated with a fatty acid. That is, an amide bond is formed between an amino group on the insulin molecule and the carboxylic acid group of the fatty acid. The amino group may be the alpha-amino group of an N-terminal amino acid of the insulin molecule, or may be the epsilon-amino group of a lysine residue of the insulin molecule. An insulin molecule of the present disclosure may be acylated at one or more of the three amino groups that are present in wild-type human insulin or may be acylated on lysine residue that has been introduced into the wild-type human insulin sequence. In certain embodiments, an insulin molecule may be acylated at position B1. In certain embodiments, an insulin molecule may be acylated at position B29. In certain embodiments, the fatty acid is selected from myristic acid (C14), pentadecylic acid (C15), palmitic acid (C16), heptadecylic acid (C17) and stearic acid (C18). For example, insulin detemir (LEVEMIR) is a long acting insulin mutant in which Thr^{B30} has been deleted, and a C14 fatty acid chain (myristic acid) has been attached to Lys^{B29}.

In some embodiments, the N-terminus of the A-peptide, the N-terminus of the B-peptide, the epsilon-amino group of Lys at position B29 or any other available amino group in an insulin molecule of the present disclosure is covalently linked to a fatty acid moiety of general formula:

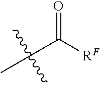

wherein R^{F} is hydrogen or a C₁₋₃₀ alkyl group. In some embodiments, R^{F} is a C₁₋₂₀ alkyl group, a C₃₋₁₉ alkyl group, a C₅₋₁₈ alkyl group, a C₆₋₁₇ alkyl group, a C₈₋₁₆ alkyl group, a C₁₀₋₁₅ alkyl group, or a C₁₂₋₁₄ alkyl group. In certain embodiments, the insulin polypeptide is conjugated to the moiety at the A1 position. In certain embodiments, the insulin polypeptide is conjugated to the moiety at the B1 position. In certain embodiments, the insulin polypeptide is conjugated to the moiety at the epsilon-amino group of Lys at position B29. In certain embodiments, position B28 of the insulin molecule is Lys and the epsilon-amino group of Lys^{B28} is conjugated to the fatty acid moiety. In certain embodiments, position B3 of the insulin molecule is Lys and the epsilon-amino group of Lys^{B3} is conjugated to the fatty acid moiety. In some embodiments, the fatty acid chain is 8-20 carbons long. In some embodiments, the fatty acid is octanoic acid (C8), nonanoic acid (C9), decanoic acid (C10), undecanoic acid (C11), dodecanoic acid (C12), or tridecanoic acid (C13). In certain embodiments, the fatty acid is myristic acid (C14), pentadecanoic acid (C15), palmitic acid (C16), heptadecanoic acid (C17), stearic acid (C18), nonadecanoic acid (C19), or arachidic acid (C20).

In certain embodiments, an insulin molecule of the present disclosure comprises the mutations and/or chemical modifications of one of the following insulin molecules: Lys^{B28}Pro^{B29}-human insulin (insulin lispro), Asp^{B28}-human insulin (insulin aspart), Lys^{B3}Glu^{B29}-human insulin (insulin glulisine), Arg^{B31}Arg^{B32}-human insulin (insulin glargine), N^{εB29}-myristoyl-des(B30)-human insulin (insulin detemir), Ala^{B26}-human insulin, Asp^{B1}-human insulin, Arg^{A0}-human insulin, Asp^{B1}Glu^{B13}-human insulin, Gly^{A21}-human insulin, Gly^{A21}Arg^{B31}Arg^{B32}-human insulin, Arg^{A0}Arg^{B31}Arg^{B32}-human insulin, Arg^{A0}Gly^{A21}Arg^{B31}Arg^{B32}-human insulin, des(B30)-human insulin, des(B27)-human insulin, des(B28-B30)-human insulin, des(B1)-human insulin, des(B1-B3)-human insulin.

In various embodiments, the conjugate may include an insulin sensitizer (i.e., a drug which potentiates the action of insulin). Drugs which potentiate the effects of insulin include biguanides (e.g., metformin) and glitazones. The first glitazone drug was troglitazone which turned out to have severe side effects. Second generation glitazones include pioglitazone and rosiglitazone which are better tolerated although rosiglitazone has been associated with adverse cardiovascular events in certain trials.

In various embodiments, a conjugate may include an insulin secretagogue (i.e., a drug which stimulates insulin secretion by beta cells of the pancreas). For example, in various embodiments, a conjugate may include a sulfonylurea. Sulfonylureas stimulate insulin secretion by beta cells of the pancreas by sensitizing them to the action of glucose. Sulfonylureas can, moreover, inhibit glucagon secretion and sensitize target tissues to the action of insulin. First generation sulfonylureas include tolbutamide, chlorpropamide and carbutamide. Second generation sulfonylureas which are active at lower doses include glipizide, glibenclamide, gliclazide, glibornuride and glimepiride. In various embodiments, a conjugate may include a meglitinide. Suitable meglitinides include nateglinide, mitiglinide and repaglinide. Their hypoglycemic action is faster and shorter than that of sulfonylureas. Other insulin secretagogues include glucagon-like peptide 1 (GLP-1) and GLP-1 analogs (i.e., a peptide with GLP-1 like bioactivity that differs from GLP-1 by 1-10 amino acid substitutions, additions or deletions and/or by a chemical modification). GLP-1 reduces food intake by inhibiting gastric emptying, increasing satiety through central actions and by suppressing glucagon release. GLP-1 lowers plasma glucose levels by increasing pancreas islet cell proliferation and increases insulin production following food consumption. GLP-1 may be chemically modified, e.g., by lipid conjugation as in liraglutide to extend its in vivo half-life. Yet other insulin secretagogues include exendin-4 and exendin-4 analogs (i.e., a peptide with exendin-4 like bioactivity that differs from exendin-4 by 1-10 amino acid substitutions, additions or deletions and/or by a chemical modification). Exendin-4, found in the venom of the Gila Monster, exhibits GLP-1 like bioactivity. It has a much longer half-life than GLP-1 and, unlike GLP-1, it can be truncated by 8 amino acid residues at its N-terminus without losing bioactivity. The N-terminal region of GLP-1 and exendin-4 are almost identical, a significant difference being the second amino acid residue, alanine in GLP-1 and glycine in exendin-4, which gives exendin-4 its resistance to in vivo digestion. Exendin-4 also has an extra 9 amino acid residues at its C-terminus as compared to GLP-1. Mann et al. Biochem. Soc. Trans. 35:713-716, 2007 and Runge et al., Biochemistry 46:5830-5840, 2007 describe a variety of GLP-1 and exendin-4 analogs which may be used in a conjugate of the present disclosure. The short half-life of GLP-1 results from enzymatic digestion by dipeptidyl peptidase IV (DPP-IV). In certain embodiments, the effects of endogenous GLP-1 may be enhanced by administration of a DPP-IV inhibitor (e.g., vildagliptin, sitagliptin, saxagliptin, linagliptin or alogliptin).

In various embodiments, a conjugate may include amylin or an amylin analog (i.e., a peptide with amylin like bioactivity that differs from amylin by 1-10 amino acid substitutions, additions or deletions and/or by a chemical modification). Amylin plays an important role in glucose regulation (e.g., see Edelman and Weyer, Diabetes Technol. Ther. 4:175-189, 2002). Amylin is a neuroendocrine hormone that is co-secreted with insulin by the beta cells of the pancreas in response to food intake. While insulin works to regulate glucose disappearance from the bloodstream, amylin works to help regulate glucose appearance in the bloodstream from the stomach and liver. Pramlintide acetate (SYMLIN®) is an exemplary amylin analog. Since native human amylin is amyloidogenic, the strategy for designing pramlintide involved substituting certain residues with those from rat amylin, which is not amyloidogenic. In particular, proline residues are known to be structure-breaking residues, so these were directly grafted from the rat sequence into the human sequence. Glu-10 was also substituted with an asparagine.

In various embodiments, a pre-conjugated drug may contain one or more reactive moieties (e.g., carboxyl or reactive ester, amine, hydroxyl, aldehyde, sulfhydryl, maleimidyl, alkynyl, azido, etc. moieties). As discussed below, these reactive moieties may, in certain embodiments, facilitate the conjugation process. Specific examples include peptidic drugs bearing alpha-terminal amine and/or epsilon-amine lysine groups. It will be appreciated that any of these reactive moieties may be artificially added to a known drug if not already present. For example, in the case of peptidic drugs a suitable amino acid (e.g., a lysine) may be added or substituted into the amino acid sequence. In addition, as discussed in more detail below, it will be appreciated that the conjugation process may be controlled by selectively blocking certain reactive moieties prior to conjugation.

### Conjugate Frameworks

This section describes some exemplary conjugate frameworks, which are also disclosed in WO2010088294, which is incorporated herein in its entirety, and which may be used to attach the saccharide to the drug. Different combinations of frameworks and saccharides conjugated to particular drugs are expected to provide drug-saccharide conjugates with different degrees of ability to bind human MRC1 and/or be uptaken by target cells that express the human MRC1, including human macrophages.

In various embodiments, a conjugate may have the general formula (I):

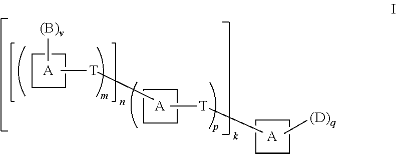

wherein:
each occurrence of

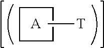

represents a potential branch within the conjugate;
each occurrence of

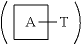

represents a potential repeat within a branch of the conjugate;
each occurrence of

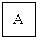

is independently a covalent bond, a carbon atom, a heteroatom, or an optionally substituted group selected from the group consisting of acyl, aliphatic, heteroaliphatic, aryl, heteroaryl, and heterocyclic;
each occurrence of T is independently a covalent bond or a bivalent, straight or branched, saturated or unsaturated, optionally substituted C₁₋₃₀ hydrocarbon chain wherein one or more methylene units of T are optionally and independently replaced by —O—, —S—, —N(R)—, —C(O)—, —C(O)O—, —OC(O)—, —N(R)C(O)—, —C(O)N(R)—, —S(O)—, —S(O)₂—, —N(R)SO₂—, —SO₂N(R)—, a heterocyclic group, an aryl group, or a heteroaryl group; each occurrence of R is independently hydrogen, a suitable protecting group, or an acyl moiety, arylalkyl moiety, aliphatic moiety, aryl moiety, heteroaryl moiety, or heteroaliphatic moiety;
—B is -T-L^{B}-X;
each occurrence of X is independently a ligand;
each occurrence of L^{B} is independently a covalent bond or a group derived from the covalent conjugation of a T with an X;
-D is -T-L^{D}-W;
each occurrence of W is independently a drug;
each occurrence of L^{D} is independently a covalent bond or a group derived from the covalent conjugation of a T with a W;
k is an integer from 1 to 12, inclusive;
q is an integer from 1 to 4, inclusive;
each occurrence of p is independently an integer from 1 to 5, inclusive; and
each occurrence of n is independently an integer from 0 to 5, inclusive; and
each occurrence of m is independently an integer from 1 to 5, inclusive; and
each occurrence of v is independently an integer from 0 to 5, inclusive, with the proviso that within each k-branch at least one occurrence of n is ≧1 and at least one occurrence of v is ≧1.

It is to be understood that general formula (I) (and other formulas herein) does not expressly list every hydrogen. For example, if the central

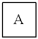

is a C₆ aryl group and k+q<6 it will be appreciated that the open position(s) on the C₆ aryl ring include a hydrogen.

In general, it will be appreciated that each occurrence of

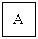

represents a potential branching node and that the number of branches at each node are determined by the values of k for the central

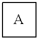

and n for non-central occurrences of

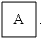

One of ordinary skill will appreciate that because each occurrence of n may be an integer from 0 to 5, the present disclosure contemplates linear, branched, and hyperbranched (e.g., dendrimer-like) embodiments of these conjugates. The proviso which requires that within each k-branch at least one occurrence of n is ≧1 and at least one occurrence of v is ≧1 ensures that every conjugate includes at least one occurrence of B (i.e., a ligand).

In certain embodiments, each occurrence of

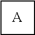

in a p-bracketed moiety is substituted by a number of n-bracketed moieties corresponding to a value of n≧1. For example, when k=2 and p=2 in both k-branches, the conjugate may be of the formula (Ia):

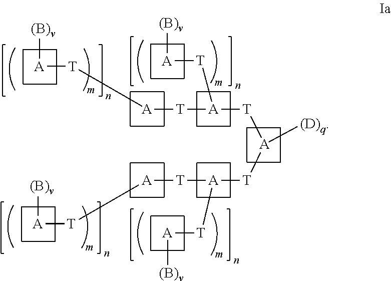

In other embodiments, only terminal occurrences of

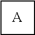

in a p-bracketed moiety are substituted by a number of n-bracketed moieties corresponding to a value of n≧1. For example, when k=2 and p=2 in both k-branches (and n=0 for the first p-bracketed moiety in both k-branches), the conjugate may be of the formula (Ib):

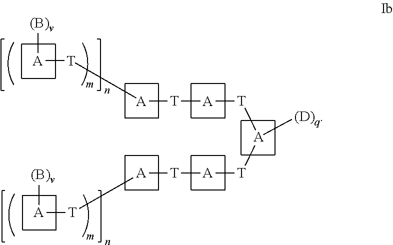

In certain embodiments, each occurrence of

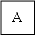

in an m-bracketed moiety is substituted by a number of B moieties corresponding to the value of v≧1. For example, when k=2, each occurrence of p=1, and each occurrence of m=2, the conjugate may be of the formula (Ic):

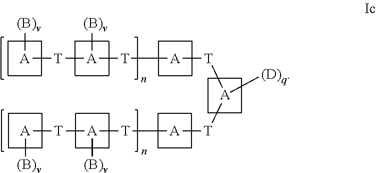

In other embodiments, only terminal occurrences of

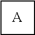

in an m-bracketed moiety are substituted by a number of B moieties corresponding to a value of v≧1. For example, when k=2, each occurrence of p=1, and each occurrence of m=2 (and v=0 for the first m-bracketed moiety in each n-branch), the conjugate may be of the formula (Id):

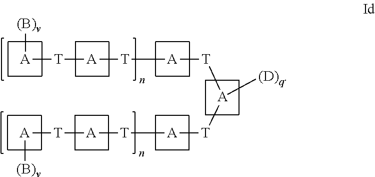

By way of further example, when q=1 and n=1 in both k-branches of the previous formula, the conjugate may be of the formula (Ie):

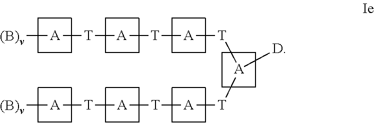

Alternatively, when q=1 and n=2 in both k-branches of the previous formula, the conjugate may be of the formula (If):

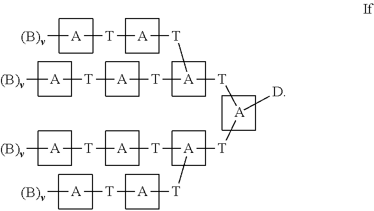

In various embodiments, the present disclosure also provides conjugates which include ligands and/or a drug which is non-covalently bound to a conjugate framework.

For example, in some embodiments, the present disclosure provides conjugates of any of the foregoing formulas, wherein:
each of

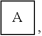

T, D, k, q, p, n, m and v is defined as described above and herein;
—B is -T-LRP^{B}-X;
each occurrence of X is independently a ligand; and
each occurrence of LRP^{B} is independently a ligand-receptor pair which forms a non-covalent bond between T and X with a dissociation constant in human serum of less than 1 pmol/L.

In yet other embodiments, the present disclosure provides conjugates of any of the foregoing formulas, wherein:
each of

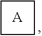

T, B, k, q, p, n, m and v is defined as described above and herein;
-D is -T-LRP^{D}-W;
each occurrence of W is independently a drug; and
each occurrence of LRP^{D} is independently a ligand-receptor pair which forms a non-covalent bond between T and W with a dissociation constant in human serum of less than 1 pmol/L.

In other embodiments, the present disclosure provides conjugates of any of the foregoing formulas wherein:
each of

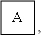

T, k, q, p, n, m and v is defined as described above and herein;
—B is -T-LRP^{B}-X;
each occurrence of X is independently a ligand;
each occurrence of LRP^{B} is independently a ligand-receptor pair which forms a non-covalent bond between T and X with a dissociation constant in human serum of less than 1 pmol/L;
-D is -T-LRP^{D}-W;
each occurrence of W is independently a drug; and each occurrence of LRP^{D} is independently a ligand-receptor pair which forms a non-covalent bond between T and W with a dissociation constant in human serum of less than 1 pmol/L.

In another aspect, a conjugate may have the general formula (II):

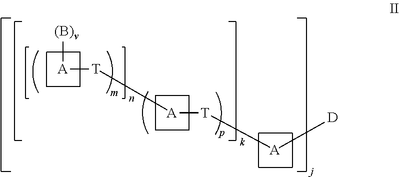

wherein

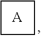

B, T, D, v, m, n, p, and k are as defined and described herein, and j is an integer from 1 to 4 inclusive. Conjugates of formula (II) may have multiple sites of conjugation of ligand to drug (i.e., where two or more ligands are conjugated to a single drug molecule via different sites on the drug, e.g., different amino acids in a biomolecular drug). It will be appreciated that, when q is 1, the subgenera described above (i.e., formulae (Ia)-(If)) apply to conjugates of formula (II) when j is 1. Likewise, similar subgenera can be contemplated by one skilled in the art for conjugates wherein j is 2, 3, or 4.

For purposes of exemplification and for the avoidance of confusion it is to be understood that an occurrence of

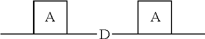

in a conjugate of formula (II) (i.e., when j is 2) could be represented as:

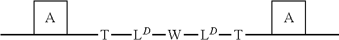

(when the drug is covalently bound to the conjugate framework) or

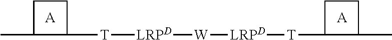

(when the drug is non-covalently bound to the conjugate framework).

### Description of Exemplary Groups

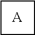

(Node)

In certain embodiments, each occurrence of

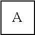

is independently an optionally substituted group selected from the group consisting of acyl, aliphatic, heteroaliphatic, aryl, heteroaryl, and heterocyclic. In some embodiments, each occurrence of

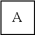

is the same. In some embodiments, the central

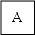

is different from all other occurrences of

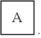

In certain embodiments, all occurrences of

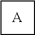

are the same except for the central

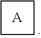

In some embodiments,

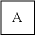

is an optionally substituted aryl or heteroaryl group. In some embodiments,

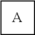

is 6-membered aryl. In certain embodiments,

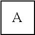

is phenyl.

In certain embodiments,

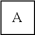

is a heteroatom selected from N, O, or S. In some embodiments,

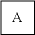

is nitrogen atom. In some embodiments,

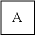

is an oxygen atom. In some embodiments,

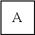

is sulfur atom. In some embodiments,

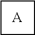

is a carbon atom.

### T (Spacer)

In certain embodiments, each occurrence of T is independently a bivalent, straight or branched, saturated or unsaturated, optionally substituted C₁₋₂₀ hydrocarbon chain wherein one or more methylene units of T are optionally and independently replaced by —O—, —S—, —N(R)—, —C(O)—, —C(O)O—, —OC(O)—, —N(R)C(O)—, —C(O)N(R)—, —S(O)—, —S(O)₂—, —N(R)SO₂—, —SO₂N(R)—, a heterocyclic group, an aryl group, or a heteroaryl group. In certain embodiments, one, two, three, four, or five methylene units of T are optionally and independently replaced. In certain embodiments, T is constructed from a C₁₋₁₀, C₁₋₈, C₁₋₆, C₁₋₄, C₂₋₁₂, C₄₋₁₂, C₆₋₁₂, C₈₋₁₂, or C₁₀₋₁₂ hydrocarbon chain wherein one or more methylene units of T are optionally and independently replaced by —O—, —S—, —N(R)—, —C(O)—, —C(O)O—, —OC(O)—, —N(R)C(O)—, —C(O)N(R)—, —S(O)—, —S(O)₂—, —N(R)SO₂—, —SO₂N(R)—, a heterocyclic group, an aryl group, or a heteroaryl group. In some embodiments, one or more methylene units of T is replaced by a heterocyclic group. In some embodiments, one or more methylene units of T is replaced by a triazole moiety. In certain embodiments, one or more methylene units of T is replaced by —C(O)—. In certain embodiments, one or more methylene units of T is replaced by —C(O)N(R)—. In certain embodiments, one or more methylene units of T is replaced by —O—.

In particular embodiments, T may be structure

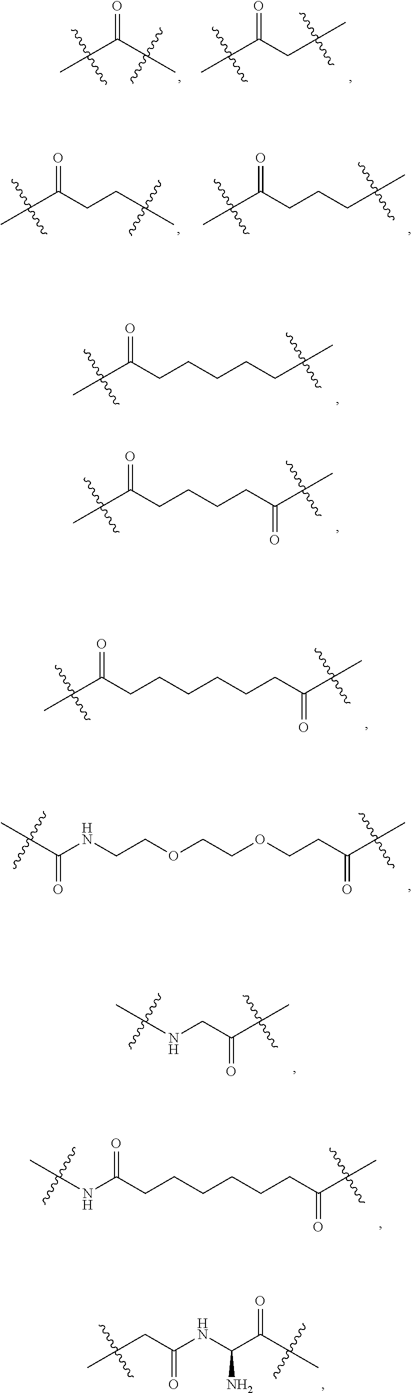

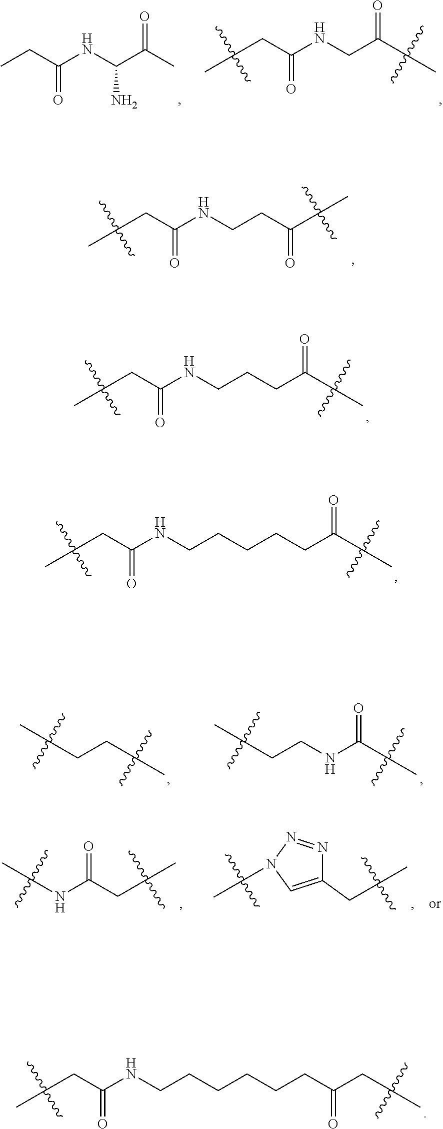

In certain embodiments, each occurrence of T is the same.

In certain embodiments, each occurrence of T (outside groups B and D) is a covalent bond and the conjugate is of the general formula (IV) or (V):

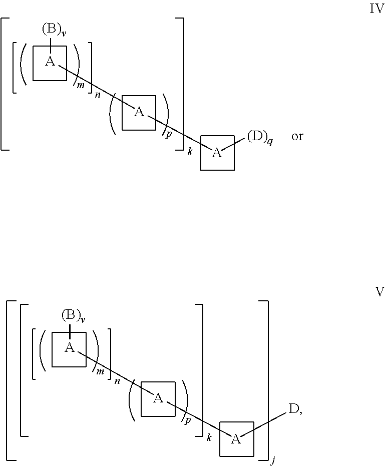

wherein

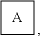

B, D, v, m, n, p, k, and j are as defined and described herein.

In certain embodiments of general formulae (IV) and (V), each occurrence of

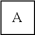

except for the central

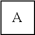

is a covalent bond, each occurrence of v=1, and the conjugate is of the formula (VI) or (VII):

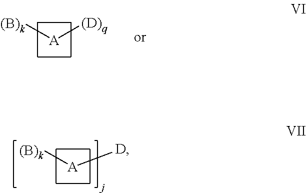

wherein

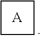

B, D, q, k, and j are as defined and described herein.

In certain such embodiments for formula (VI), k=1 and q=1.

In other embodiments, k=2 and q=1.

In other embodiments, k=3 and q=1.

In other embodiments, k=1 and q=2.

In other embodiments, k=2 and q=2.

In certain such embodiments for formula (VII), k=1 and j=2.

In other embodiments, k=2 and j=2.

In other embodiments, k=3 and j=2.

In other embodiments, k=1 and j=1.

In other embodiments, k=2 and j=1.

In other embodiments, k=3 and j=1.

In other embodiments, k=1 and j=3.

In other embodiments, k=2 and j=3.

In other embodiments, k=3 and j=3.

The following examples are intended to promote a further understanding of the present invention.

### Example 1

MRC1 is responsible for the uptake of insulin-saccharide conjugates in rat macrophage cells.

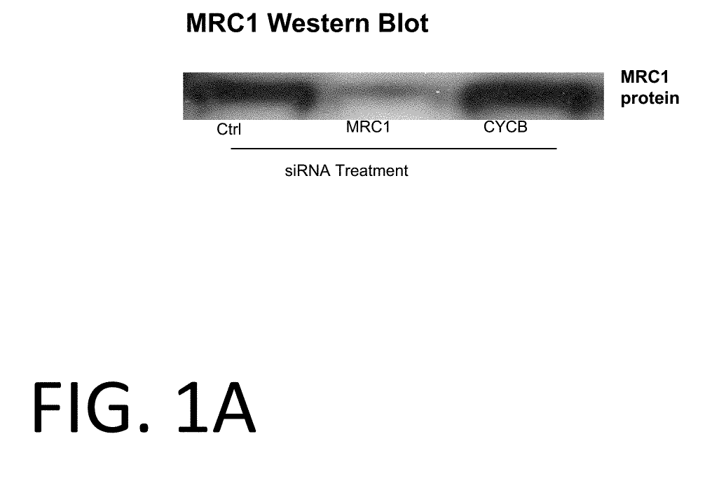

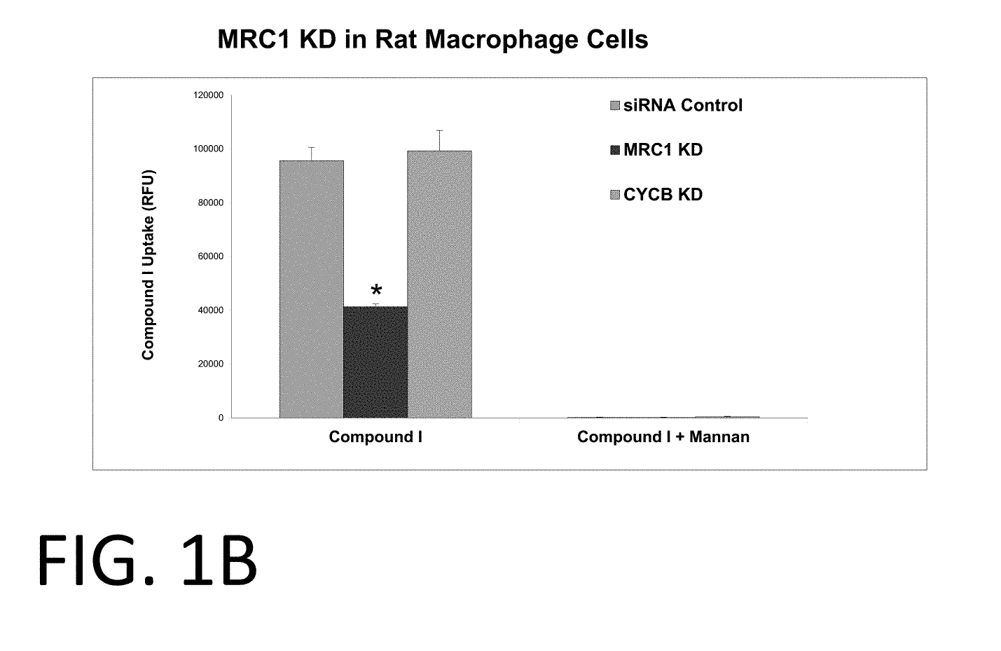

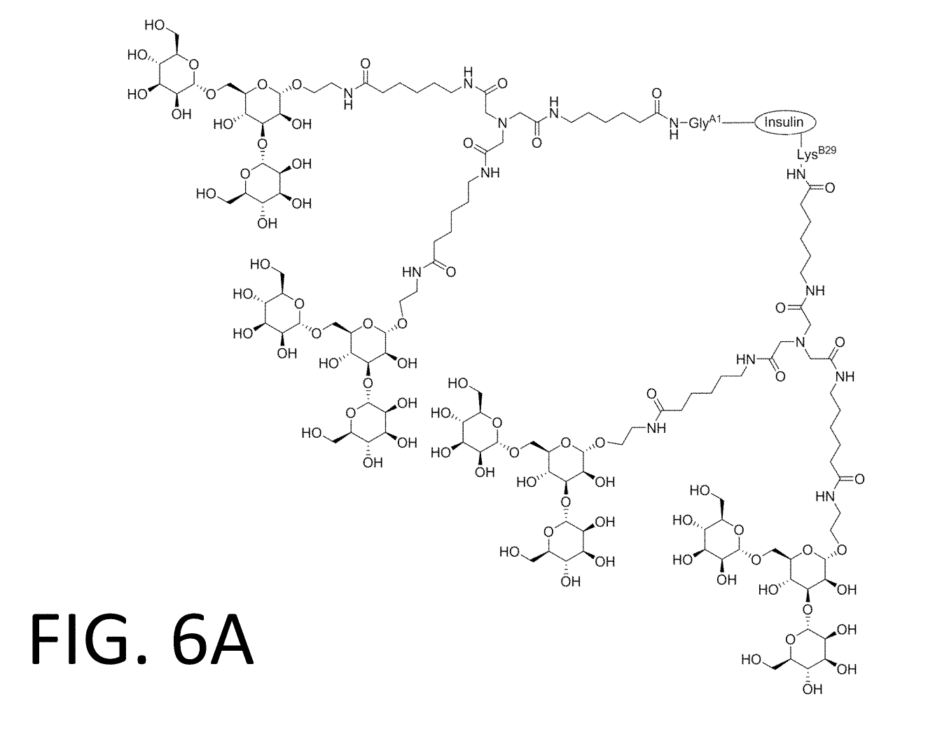

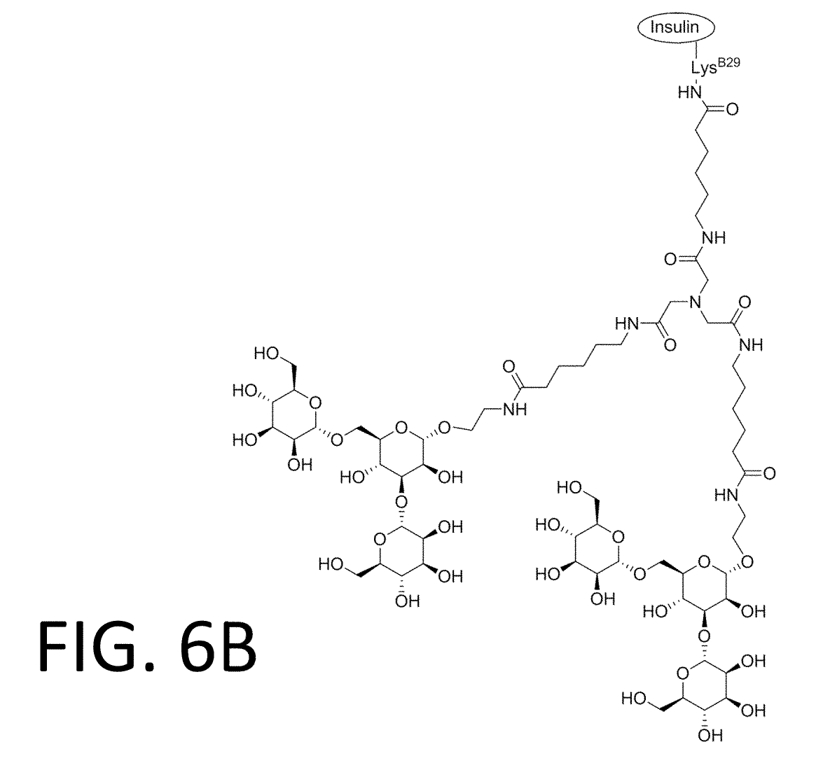

Rat macrophage cell line, NR8383, can effectively take up insulin-saccharide conjugates in a mannan dependent manner. Rat macrophage cells, NR8383 (ATCC #CRL2192), were culture in F12K medium containing 15% FBS. Cells were plated in 24-well collagen coated plates to reach 80% confluency. Cells were washed with PBS and transfected with rat MRC1 siRNA, rat CYCB siRNA, or control non-targeting siRNA (siNT) (Dharmacon) using Mirus TransItTKO reagent (Mirus MIR2150) according to manufacturer's protocol. After 48 hours, IOCs' uptake was measured and cells were collected for mRNA or protein analysis. Rat MRC1 siRNA knockdown led to 65% reduction of MRC1 proteins in NR8383 cells according to Western blot analysis using a MRC1 antibody (Abcam, #ab64693) as shown in FIG. 1A. Knockdown of MRC1 resulted in 57% reduction in the uptake of 250 nM Alexa-488 labeled Compound I in NR8383 cells as shown in FIG. 1B. Cells were washed with ice cold HSSB buffer containing 1% BSA and 0.1% FBS and fixed with 4% paraformaldehyde for 20 minutes. Fluorescence of internalized Compound I was measured by high-content image analysis (ArrayScan VTI from Thermofisher). As a control, CYCB knockdown data indicate it did not affect Compound I uptake. Compound I as shown in FIG. 6A was prepared as described in WO 2010/88294 (see methods that were used to make conjugate 11-2 or TSAT-C6-Di-sub-AETM-2 (A1,B29) in Example 76, which is incorporated herein by reference).

### Example 2

MRC1 is the major receptor for the uptake of insulin-saccharide conjugates in human primary macrophages.

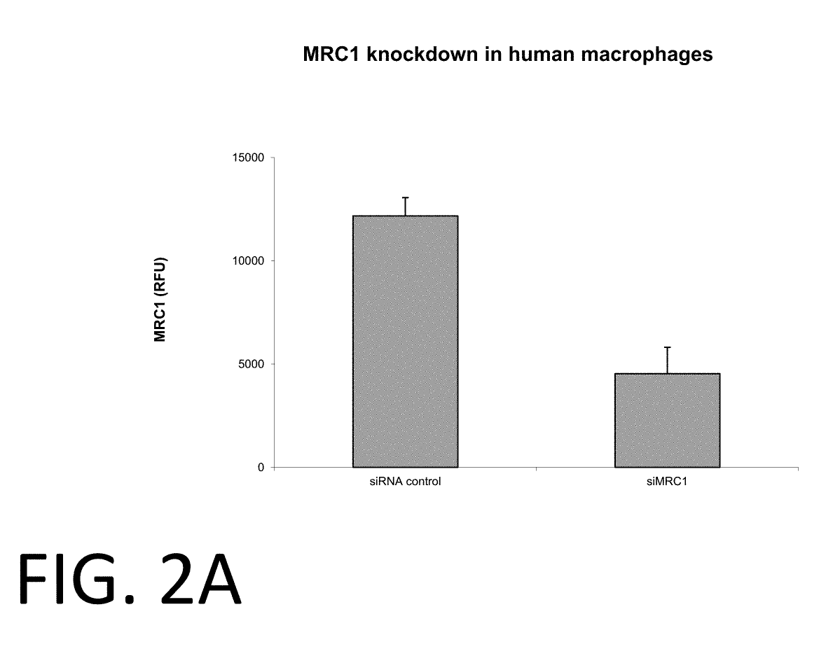

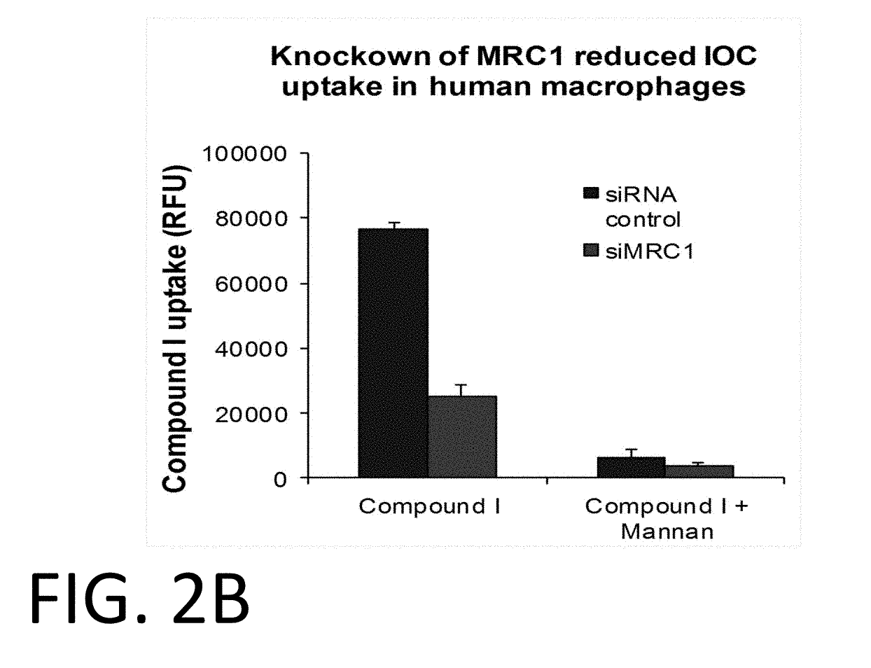

Freshly isolated PBMCs from human blood were differentiated into macrophages in RPMI1640 medium containing 10% FBS containing 50 ng/ml MCSF (R&D systems) in 3 days. Cells were further incubated with fresh differentiation medium for another 2 days. The differentiated macrophages were removed from T75 flasks and siRNA transfection was performed by electroporation using Ingenio solution (Mirus #50112) as described by manufacturer's protocol. Cells were plated into 96-well or 24-well plates and incubated in differentiation medium for 2 days for further analysis. The human macrophages can effectively take up insulin-saccharide conjugates in a mannan dependent manner similar to rat macrophage cells. Human MRC1 siRNA knockdown led to more than 60% reduction of MRC1 membrane levels measured by flow cytometry method using APC-MRC1 antibody (BD bioscience #550889) as shown in FIG. 2A. siRNA transfected cells were washed with PBS and incubated with 250 nM Alexa-488 labeled Compound I in assay buffer (HEPES buffered saline pH 7.4, 1% BSA, 0.1% HI FBS, 2 mM CaCl2+0.5 mM MgSO4) at 37° C. for 1 hour. Cells were washed with ice cold HSSB buffer containing 1% BSA and 0.1% FBS and fixed with 4% paraformaldehyde for 20 minutes. Cells were washed with PBS and PBS were added to each well and stored at 4° C. in the dark for future analysis. Fluorescence was quantified by high-content image analysis (ArrayScan VTI from Thermofisher). Knockdown of MRC1 resulted in more than 70% reduction in Alexa-488 labeled Compound I uptake in human primary macrophage cells as shown in FIG. 2B.

### Example 3

Overexpression of MRC1 in HEK293 cells increased insulin-saccharide conjugate uptake robustly.

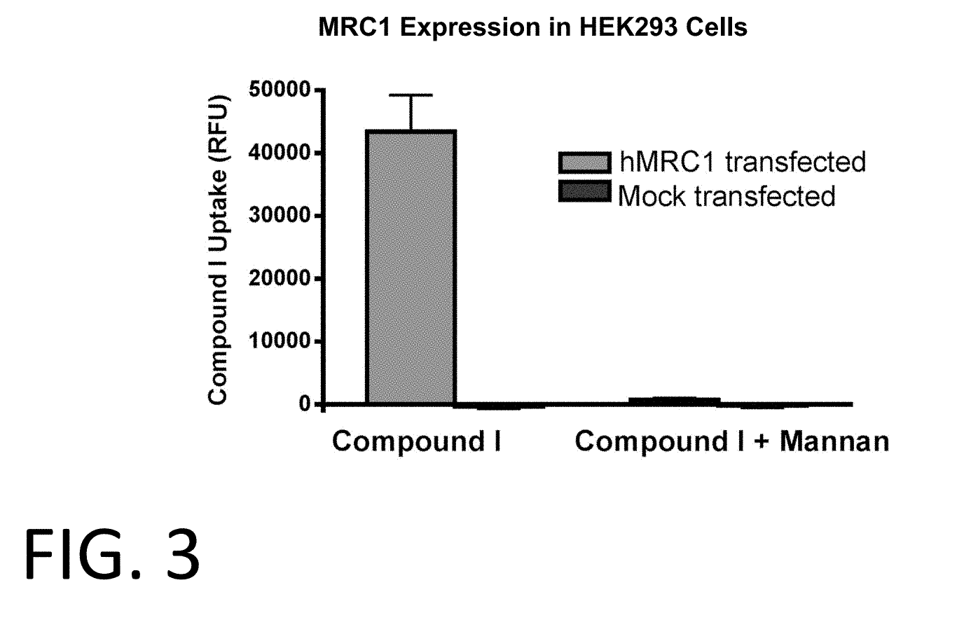

HEK293 cells transfected with an expression vector encoding human MRC1 led to overexpression of MRC1 protein. HEK293 cells were plated onto 96-well collagen coated plate in DMEM growth medium with 10% FBS. After the cells reach 90% confluency, cells were transfected with human MRC1 gene using Lipofectamine 2000. 24 hours after transfection, Compound I uptake was measured in the presence of 250 nM Alexa-488 labeled Compound I in assay buffer (HEPES buffered saline pH 7.4, 1% BSA, 0.1% HI FBS, 2 mM CaCl2 and 0.5 mM MgSO4) with or without 10 mg/ml mannan at 37° C. for 1 hour. HEK293 parental cells hardly took up any Compound I as shown in FIG. 3. However, overexpression of human MRC1 increased Compound I uptake robustly and this effect could be completely blocked by mannan.

### Example 4

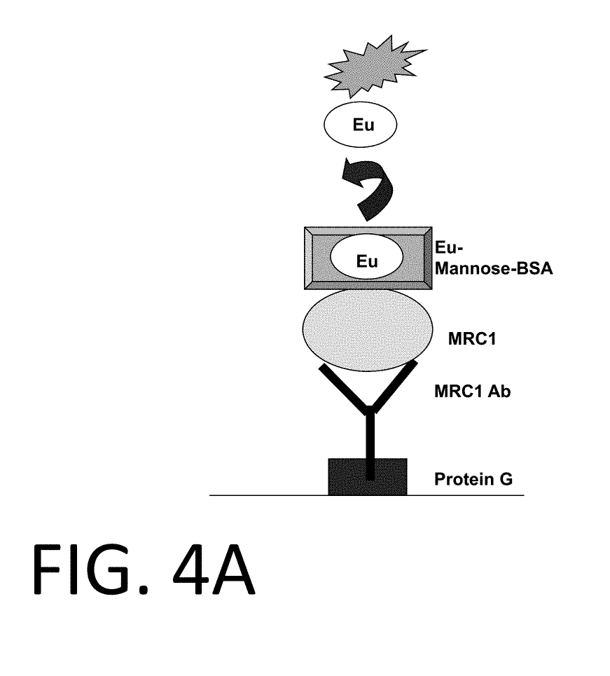

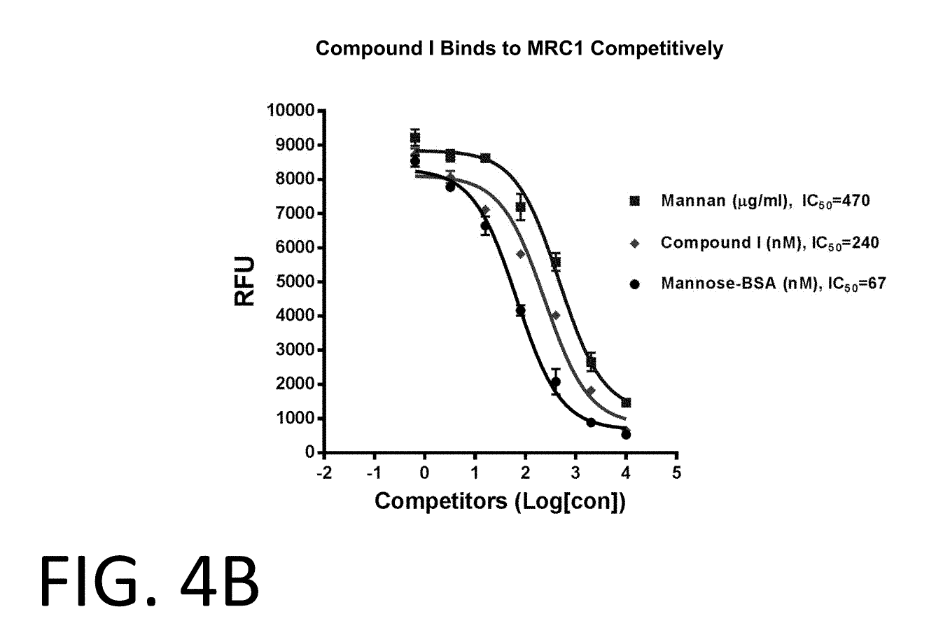

A newly developed competition assay allows measurement of insulin-saccharide conjugate binding potency to mannose receptor (FIG. 4A). Anti-MRC1 antibody binds to protein G coated plates, which then binds to MRC1 protein. Binding of Europium-labeled mannose-BSA conjugate to MRC1 is measured in the assay alone or in the presence of competitors (e.g., exemplified with insulin-saccharide conjugate Compound I) as shown in FIG. 4B. The results allow determination of insulin-saccharide conjugate binding potency.

The competition binding assay for MRC1 utilizes a ligand, mannosylated-BSA labeled with the DELFIA Eu—N1-ITC reagent, as reported in the literature. 200 ng anti-MRC1 antibody (R&D, AF2534) per well is added to a Protein G plate that had been washed three times with 100 μl of 50 mM Tris buffer, pH 7.5, 100 mM NaCl, 5 mM CaCl₂, 1 mM MgCl₂ and 0.1% Tween-20 (wash buffer). The antibody is incubated in the plate for 1 hour at room temperature with shaking. The plate is washed with wash buffer 3-5 times followed by addition of 100 ng/well his-tagged human macrophage mannose receptor MRC1 (R&D systems, 2534-MR) in PBS plus protease inhibitor and shake for 1 hour at room temperature. Repeat wash plate 3 times with above wash buffer and then add Eu-mannosylated-BSA (0.1 nM final concentration) and its competitors (10 mg/ml mannan), 1 μM Compound I, mannose BSA) and their 1:5 dilutions in binding buffer (50 mM Tris, pH 7.5, 100 mM NaCl, 15 mM CaCl2, 5 mM MgCl2, 1% BSA plus protease inhibitor cocktail) for 2 hours at room temperature with shaking. Wash plate 3 times with cold wash buffer. Perkin Elmer Eu-inducer reagent (PerkinElmer #4013-0010; 100 μl per well) is added and incubated for 15 minutes at room temperature prior to detection of the Eu signal on Envision mono chrometer (Excitation=340 nm: Emission=615 nm).

### Example 5

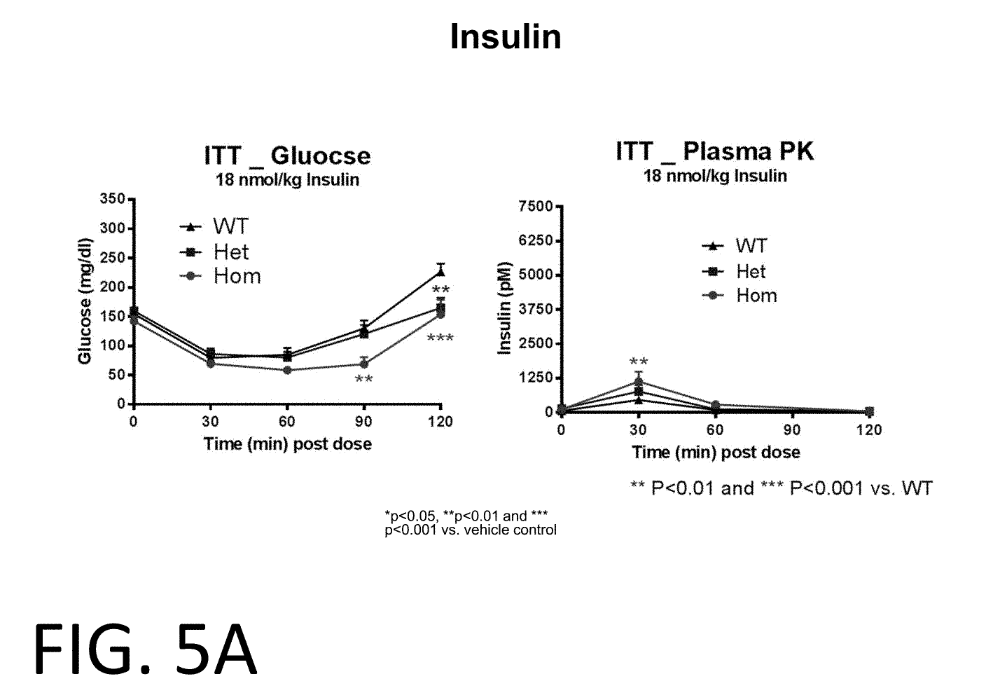

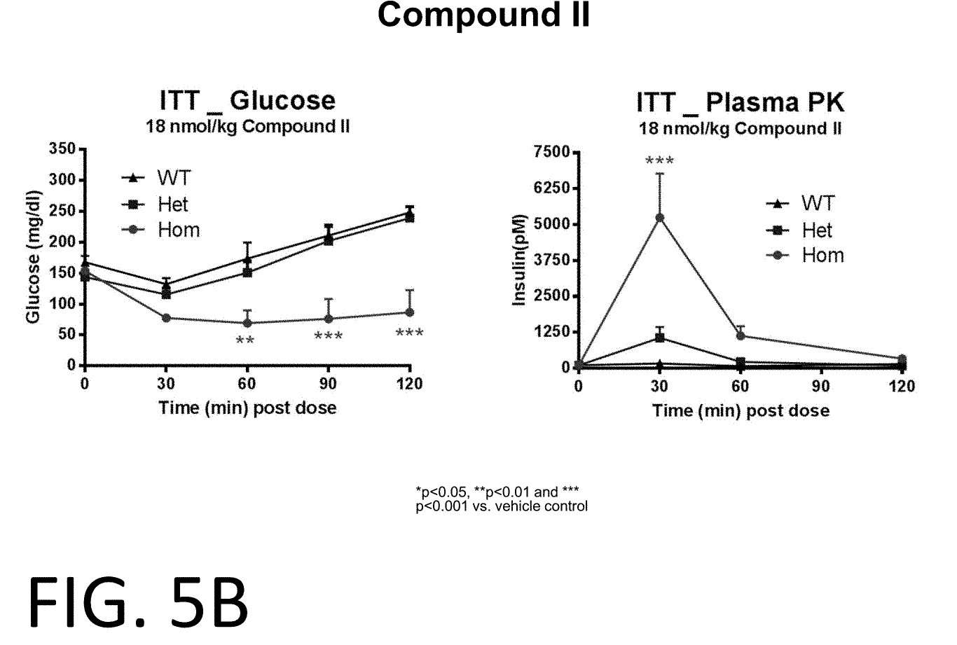

Higher levels of the plasma levels of exemplary insulin saccharide conjugate Compound II in MRC1 KO mice correlate with increased glucose lowering (FIG. 5A and FIG. 5B).

Plasma glucose and Compound II levels were measured during an insulin tolerance test. Wildtype, MRC1 heterzygous (Het) and homozygous (Hom) knockout (KO) mice were fasted for two hours before single bolus subcutaneously (s.c.) injection of insulin or Compound II (18 nmol/kg) at time 0. Glucose was measured by glucometer at time 0, 30, 60, 90 and 120 min. Plasma was collected at time 0, 30, 60 and 120 minutes and insulin levels were determined using Iso-insulin ELISA kit (Mercodia/Iso-insulin ELISA). Compound II as shown in FIG. 6B was prepared in accordance with methods that are disclosed in WO 2010/88294 (see methods that were used to make conjugate I-6 or TSAT-C6-AETM-2 (B29) in Example 20).

MRC1 Hom KO mice have significantly higher levels of the plasma Compound II, correlating with robust increase in glucose lowering potency (comparable to insulin potency at same dose) as compared to wildtype or Het mice. The results show MRC1 plays a predominant role in insulin-saccharide conjugate clearance in mice.

### Example 6

Alexa488-Compound I is prepared by reacting 276 nmol Compound I (an exemplary insulin-saccharide conjugate whose structure is shown in FIG. 6) with 1 mg Alexa488 Succinimidyl Ester (Invitrogen) in 667 μl 0.1M sodium bicarbonate buffer, pH=8.3, with constant stirring for 1 hour at room temperature. Labeled Compound I is separated from unreacted dye using 6 kDa NMWCO desalting columns (Pierce). Fractions containing Compound I (as determined by absorbance at 280 nM) are pooled and concentrated using 3000 Da NMWCO centrifugal concentrators (Millipore). Concentration of Compound I is determined using a BCA total protein assay (Pierce).

HEK293 cells expressing the human MRC1 are cultured in coated flasks. For uptake experiments, the cells are seeded in coated 96 well plates and allowed to reach confluence. Cells are washed 1× with PBS and incubated for 1 hour (at 37° C., 5% CO₂) with varying concentrations of Alexa488 Compound I (in HEPES buffered saline [pH=7.4] containing 1% BSA, 0.1% HI FBS, 2 mM Ca²⁺, and 0.5 mM Mg²⁺). Each condition is carried out in triplicate. Each concentration of Alexa488-Compound I is tested with and without the presence of 10 mg/mL mannan, which is known to block binding by the mannose receptor. Cells are washed and then resuspended in 1% paraformaldehyde in PBS and stored at 4° C. in the dark until analysis using ArrayScan VTI.

### Example 7

The uptake of Alexa488-Compound I is measured, as described above, in the presence of various sugars known to have varying affinities for the mannose receptor. The HEK293 cells expressing the human MRC1 are incubated with a constant concentration of Alexa488-Compound I (250 nM, chosen because this concentration lies on the concentration dependent portion of the Alexa488-Compound I uptake curve) and varying concentrations of α-methyl mannose (α-MM), glucose, and galactose.

Sugars with greater affinity for the receptor involved in Alexa488-Compound I uptake will cause a decrease in Alexa488-Compound I uptake at lower concentrations than sugars with a lower affinity.

### Example 8

Ovalbumin and zymosan are known ligands of MRC1. Therefore, FITC-ovalbumin or -zymosan may be used as a marker of uptake by this receptor. HEK293 cells expressing the human MRC1 are incubated, as described above, with a fixed concentration of FITC-ovalbumin or -zymosan (250 nM, on the concentration dependent portion of it uptake curve) in the presence of varying amounts of unlabeled conjugates. It is expected that conjugates with greater affinity for MRC1 (the pathway by which FITC-ovalbumin or -zymosan is internalized) will inhibit FITC-ovalbumin or -zymosan uptake at lower concentrations than those with a lower affinity for the human MRC1.

### Example 9

HEK293 cells expressing the human MRC1 are incubated, as described above, with a constant concentration (250 nM) of FITC-ovalbumin and various mixtures of Compound II and RHI at varying concentrations. The data is expected to show that the ability of Compound II to inhibit FITC-ovalbumin uptake is independent of the amount of RHI present. This indicates that the insulin receptor pathway does not play a role in the ability of the conjugate to be taken up by the human MRC1 (i.e., there is no cooperativity between the two pathways).

### Example 10

Insulin Receptor Phosphorylation Assays may be performed as follows.

The insulin receptor phosphorylation assays may be performed using the commercially available Meso Scale Discovery (MSD) pIR assay (See Meso Scale Discovery, 9238 Gaithers Road, Gaitherburg, Md.). CHO cells stably expressing human IR(B) are in grown in F12 cell media containing 10% FBS and antibiotics (G418, Penicillin/Strepavidin) for at least 8 hours and then serum starved by switching to F12 media containing 0.5% BSA (insulin-free) in place of FBS for overnight growth. Cells are harvested and frozen in aliquots for use in the MSD pIR assay. Briefly, the frozen cells are plated in either 96-well (40,000 cells/well) or 384-well (10,000 cells/well) clear tissue culture plates and allowed to recover. Insulin-saccharide conjugates at the appropriate concentrations are added and the cells incubated for 8 min at 37° C. The media is aspirated and chilled MSD cell lysis buffer is added as per MSD kit instructions. The cells are lysed on ice for 40 min and the lysate then mixed for 10 minutes at room temperature. The lysate is transferred to the MSD kit pIR detection plates. The remainder of the assay is carried out following the MSD kit recommended protocol.

### Example 11

Insulin Receptor Binding Assays may be performed as follows. Two competition binding assays may be utilized to determine insulin-saccharide conjugate affinity for the human insulin receptor type B (IR(B)) against the endogenous ligand, insulin, labeled with 125[I].

Method A: IR binding assay is a whole cell binding method using CHO cells overexpressing human IR(B). The cells are grown in F12 media containing 10% FBS and antibiotics (G418, Penicillin/Strepavidin), plated at 40,000 cells/well in a 96-well tissue culture plate for at least 8 hrs. The cells are then serum starved by switching to DMEM media containing 1% BSA (insulin-free) overnight. The cells are washed twice with chilled DMEM media containing 1% BSA (insulin-free) followed by the addition of insulin-saccharide conjugate at appropriate concentration in 90 μL of the same media. The cells are incubated on ice for 60 min. The ¹²⁵[I]-insulin (10 μL) is added at 0.015 nM final concentration and incubated on ice for 4 hrs. The cells are gently washed three times with chilled media and lysed with 30 μL of Cell Signaling lysis buffer (cat #9803) with shaking for 10 min at room temperature. The lysate is added to scintillation liquid and counted to determine ¹²⁵[I]-insulin binding to IR and the titration effects of IOC molecules on this interaction.

Method B: IR binding assay is run in a scintillation proximity assay (SPA) in 384-well format using cell membranes prepared from CHO cells overexpressing human IR(B) grown in F12 media containing 10% FBS and antibiotics (G418, Penicillin/Strepavidin). Cell membranes are prepared in 50 mM Tris buffer, pH 7.8 containing 5 mM MgCl₂. The assay buffer contains 50 mM Tris buffer, pH 7.5, 150 mM NaCl, 1 mM CaCl₂, 5 mM MgCl₂, 0.1% BSA and protease inhibitors (Complete-Mini-Roche). Cell membranes are added to WGA PVT PEI SPA beads (5 mg/ml final concentration) followed by addition of insulin-saccharide conjugates at appropriate concentrations. After 5-15 min incubation at room temperature, ¹²⁵[I]-insulin is added at 0.015 nM final concentration for a final total volume of 50 μL. The mixture is incubated with shaking at room temperature for 1 to 12 hours followed by scintillation counting to determine ¹²⁵[I]-insulin binding to IR and the titration effects of insulin-saccharide conjugates on this interaction.

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

1-6. (canceled)

7: A method for selecting a drug-saccharide conjugate that exhibits decreased uptake by target cells that express a human mannose receptor, C type 1 (MRC1) in the presence of an inhibitor from a plurality of candidate drug-saccharide conjugates, comprising:
(a) providing the target cells that express the human MRC1;
(b) exposing the target cells to the plurality of candidate drug-saccharide conjugates and selecting candidate drug-saccharide conjugates that are taken up into the target cells;
(d) determining whether the uptake of the selected candidate drug-saccharide conjugate is decreased when the target cells are exposed to the selected candidate drug-saccharide conjugate and an inhibitor that binds the human MRC1; and
(e) selecting at least one candidate drug-saccharide conjugate which exhibits uptake by the target cells in the absence of the inhibitor and decreased uptake in the presence of the inhibitor to select the drug-saccharide conjugate,
wherein the target cells are HEK293 host cells that include an expression vector encoding the human MRC1 and which overexpress the human MRC1 and wherein the inhibitor is labeled with Alexa488 and having the formula

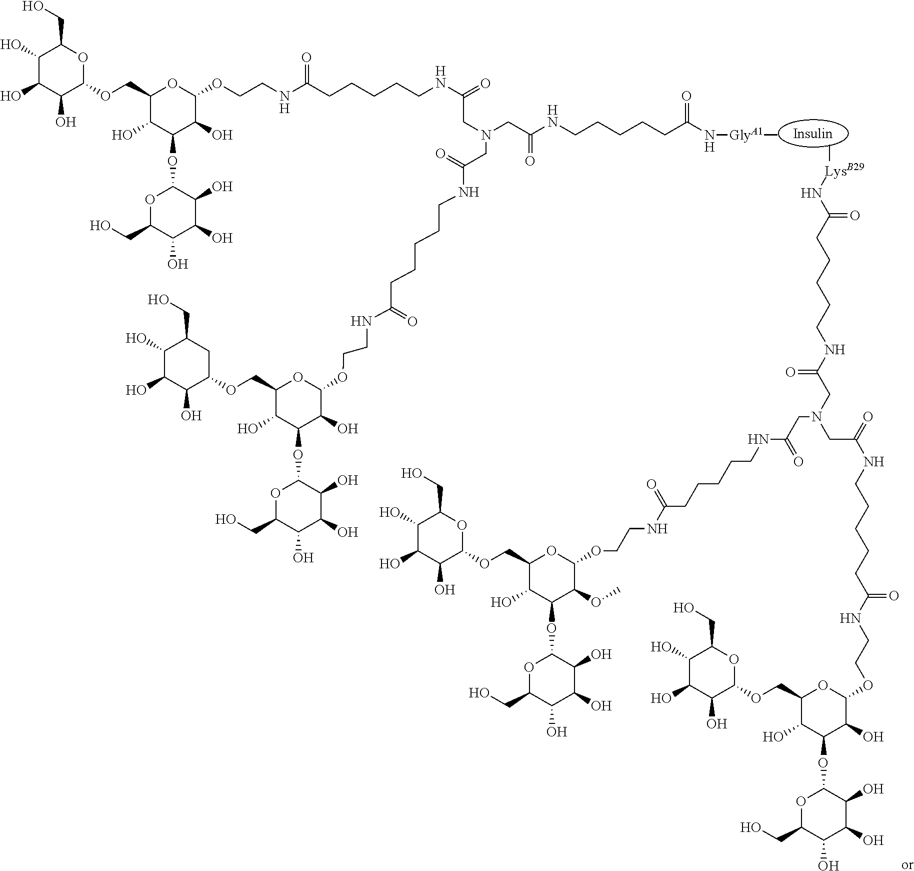

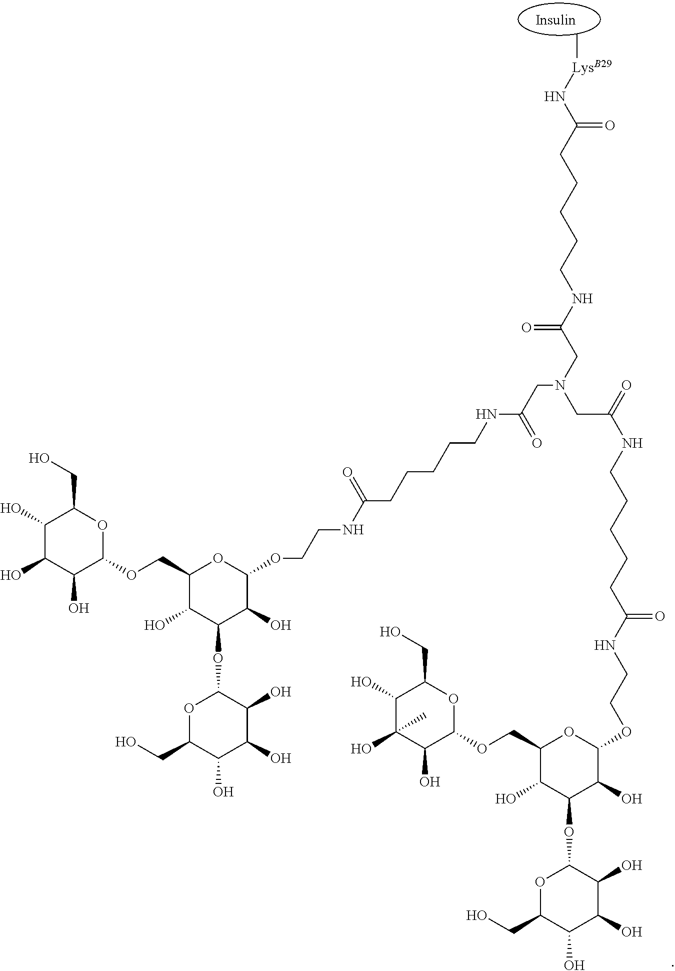

8: The method of claim 7, wherein the drug is an insulin molecule.

9-11. (canceled)

12: A method for selecting a drug-saccharide conjugate which decreases uptake of a control compound by target cells that express the human mannose receptor, C type 1 (MRC1), comprising:
(a) providing the target cells that express the human MRC1;
(b) exposing the target cells to a control compound labeled with Alexa488 and having the formula 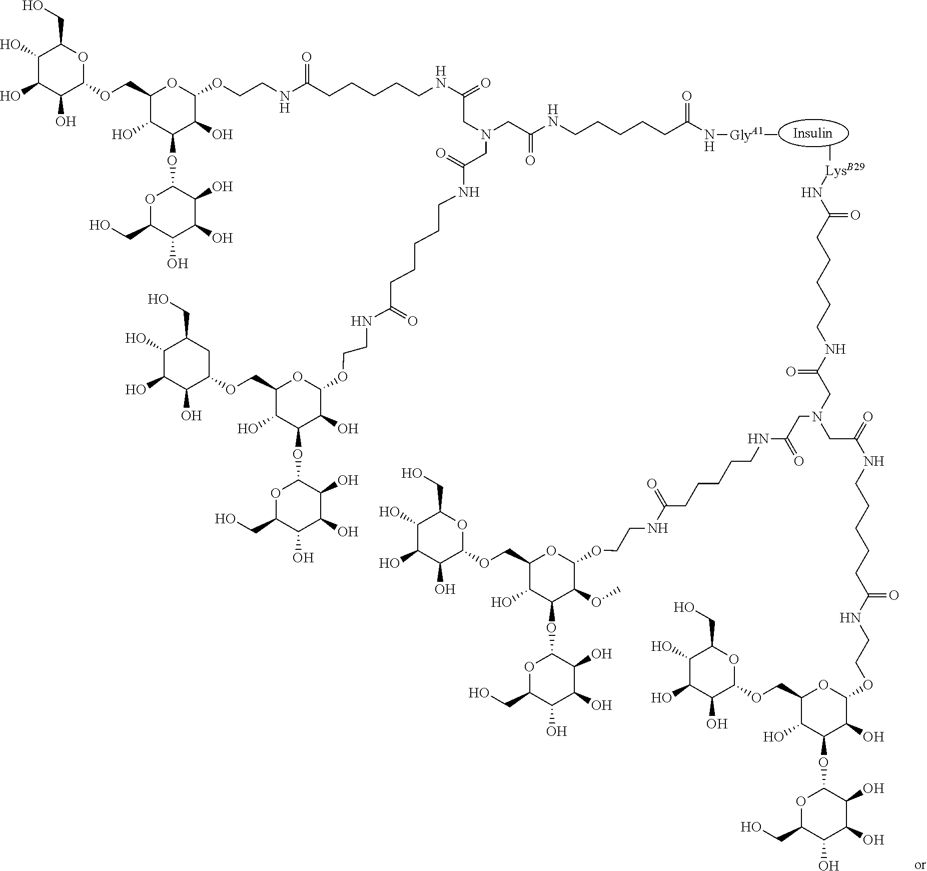 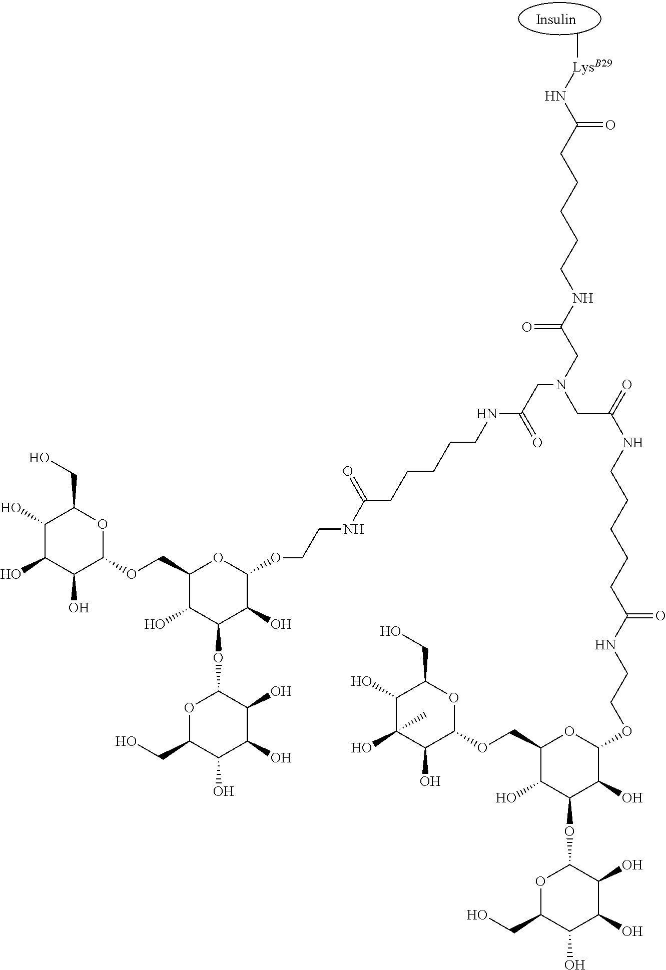
and a plurality of candidate drug-saccharide conjugates;
(c) determining whether the uptake of the control compound is decreased when the target cells are exposed to a candidate drug-saccharide conjugate; and
(e) selecting the candidate drug-saccharide conjugate which inhibits uptake of the control compound by the target cells in the to provide the drug-saccharide conjugate,
wherein the target cells are HEK293 host cells that include an expression vector encoding the human MRC1 and which overexpress the human MRC1.

13: The method of claim 12, wherein the drug is an insulin molecule.

14-17. (canceled)
